SEQUENCE LISTING (2 sequences)

SEQ ID NO: 1 - protein, 16 aa, Artificial Sequence
  Description of artificial sequence: synthetic peptide sequence
WTAVQMAVFIHNFKRK

SEQ ID NO: 2 - protein, 15 aa, Artificial Sequence
  Description of artificial sequence: synthetic peptide sequence
TAVQMAVFIHNFKRK

(19) United States
(12) Patent Application Publication
(10) Pub. No.: US 2023/0190863 A1
(43) Pub. Date: Jun. 22, 2023

(54) PHARMACEUTICAL COMPOSITIONS AND ANTI-VIRAL USES THEREOF

(71) Applicant: CODE PHARMA B.V., De Bilt (NL)

(72) Inventors: Zyon AYNI, Rehovot (IL); Eynat FINKELSHTEIN, Rehovot (IL); Esmira NAFTALI, Rehovot (IL)

(73) Assignee: CODE PHARMA B.V., De Bilt (NL)

(21) Appl. No.: 17/996,932

(22) PCT Filed: Apr. 21, 2021
(86) PCT No.: PCT/IB2021/053299

§ 371 Date: Oct. 23, 2022

Related U.S. Application Data
(60) Provisional application No. 63/013,595, filed on Apr. 22, 2020.

(51) Int. Cl. A61K 38/10; A61P 31/14; A61K 47/26; A61P 31/16

(52) U.S. Cl. CPC A61K 38/10; A61K 47/26; A61P 31/14; A61P 31/16

(57) ABSTRACT

The present invention provides pharmaceutical compositions of a hydroxyl halide of a peptide and methods of use of these compositions in treating viral infections caused by coronavirus and influenza viruses.

### CROSS REFERENCE TO RELATED APPLICATION

This international application claims priority to and the benefit of U.S. Provisional Pat. Application Serial No. 63/013,595, filed on Apr. 22, 2020, the entire contents of each of which are incorporated by reference herein.

### SEQUENCE LISTING

This application contains a Sequence Listing which has been submitted electronically in ASCII format, which is hereby incorporated by reference in its entirety. Said ASCII copy, created on Apr. 20, 2021 is named 169505-010501PCT_SL.txt and is 830 bytes in size.

### BACKGROUND OF THE INVENTION

Since its emergence in late 2019, the novel severe acute respiratory syndrome coronavirus-2 (SARS-CoV-2), identified as the etiological agent of coronavirus disease-19 (COVID-19), has caused a rapidly spreading global pandemic associated with significant morbidity and mortality and far-reaching economic and social implications. By early June 2020, more than seven million confirmed cases and more than 400,000 SARS-CoV-2-related deaths had been reported worldwide. While the majority of infected patients are asymptomatic or present with mild disease, severe disease associated with acute respiratory distress syndrome (ARDS) and multi-organ failure complicate 5-20% of COVID-19 cases. No treatment has shown robust and significant effectiveness in the treatment of mild and moderate COVID-19 cases.

Human influenza viruses cause seasonal epidemics of disease (known as the flu season). Influenza A viruses also cause flu pandemics, i.e., global epidemics of flu disease. A pandemic can occur when a novel influenza A virus emerges. For example, Influenza A (e.g. H1N1) viruses caused the pandemic that emerged in the spring of 2009. This virus, scientifically called the “A(H1N1)pdm09 virus,” and more generally called “2009 H1N1,” has continued to circulate seasonally since then. H1N1 viruses have undergone relatively small genetic changes and changes to their antigenic properties (i.e., the properties of the virus that affect immunity) over time.

Accordingly, there is a need for safe and effective treatments and therapies for COVID-19 disease, as well as other pandemics and diseases caused by coronaviruses and influenza viruses, in order to stop viral spread, infection of individuals, and loss of lives.

### SUMMARY OF THE INVENTION

The invention provides compositions and methods for treating a viral infection in a subject.

In an aspect of the invention, a pharmaceutical composition comprising a peptide consisting of amino acid sequence WTAVQMAVFIHNFKRK (SEQ ID NO: 1, INS) or a pharmaceutically acceptable salt thereof, and a pharmaceutically acceptable carrier, is provided for use in treatment of a disease caused by at least one of a coronavirus and an influenza virus. In an embodiment, the coronavirus is a human coronavirus. In an embodiment, the human coronavirus is one or more of MERS-CoV, SARS-CoV and SARS-CoV-2 viruses. In an embodiment, the disease is COVID-19. In an embodiment, the disease is influenza. In an embodiment, the influenza virus is influenza A virus.

In an embodiment of the above-delineated aspect and/or its embodiments, the pharmaceutical composition comprises from about 0.1 to about 30 mg/ml of hydrochloride salt of the peptide (INS-HCl). In an embodiment, the pharmaceutical composition comprises from about 0.1 wt% to about 15 wt% of a poloxamer having the structure of Formula I,

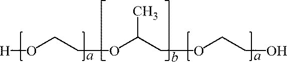

wherein the pH of the composition is between 4 to about 7.5 and wherein a is an integer from 50 to 120, and b is an integer from 15 to 40. In an embodiment, a is an integer from 60 to 90 and b is an integer from 25 to 35. In an embodiment, a=80 and b=27 (poloxamer 188). In an embodiment, the composition comprises from about 0.1 wt% to about 5 wt% of poloxamer 188. In an embodiment, the pharmaceutical composition further includes a saccharide. In an embodiment, the composition includes from about 1 wt% to about 20 wt% of the saccharide. In an embodiment, the composition includes from about 2 wt% to about 10 wt% of a saccharide. In an embodiment, the saccharide is selected from a polyol, disaccharide and polysaccharide. In an embodiment, the saccharide is selected from mannitol and lactose. In an embodiment, the mannitol is D-mannitol.

In an embodiment the pharmaceutical composition for use as delineated above includes from 5 to 15 mg/ml of INS-HCl, from about 0.1 to about 5 wt% of poloxamer 188, and from about 1 to about 10 wt% of mannitol. In another embodiment, the pharmaceutical composition for use as delineated above includes from 8 to 12 mg/ml of INS-HCl, from about 0.3 to about 1 wt% of poloxamer 188, and from about 3 to about 6 wt% of mannitol.

In an embodiment, the pharmaceutical composition for use of the above-delineated aspect and/or the embodiments thereof is administered by a route which may be subcutaneous administration, intravenous administration, and intramuscular administration. In an embodiment, the administration is by subcutaneous administration. In an embodiment, from 5 to 100 mg/day of the INS-HCl is administered. In an embodiment, from 10 to 60 mg/day of the INS-HCl is administered. In an embodiment, from 10 to 30 mg/day of the INS-HCl is administered. In an embodiment, an additional active agent is administered. In an embodiment, the active agent is an anti-viral active agent.

In another aspect of the invention, a method of treating coronavirus infection or disease is provided, in which the method involves administering to a subject in need thereof an effective amount of a pharmaceutical composition comprising a peptide comprising the amino acid sequence WTAVQMAVFIHNFKRK (SEQ ID NO: 1, INS) or a pharmaceutically acceptable salt thereof, and a pharmaceutically acceptable carrier, excipient, or diluent. In an embodiment, the coronavirus is a human coronavirus. In an embodiment, the human coronavirus is selected from MERS-CoV, SARS-CoV and SARS-CoV-2. In an embodiment, the disease is COVID-19.

In another aspect of the invention, a method of treating influenza virus infection or disease is provided, in which the method involves administering to a subject in need thereof an effective amount of a pharmaceutical composition comprising a peptide comprising the amino acid sequence WTAVQMAVFIHNFKRK (SEQ ID NO: 1, INS) or a pharmaceutically acceptable salt thereof, and a pharmaceutically acceptable carrier, excipient, or diluent. In an embodiment, the influenza virus is influenza A virus.

In an embodiment of the method of any of the above-delineated aspects and/or the embodiments thereof, the pharmaceutical composition comprises from about 0.1 to about 30 mg/ml of hydrochloride salt of the peptide (INS-HCl). In an embodiment of the method of any of the above-delineated aspects and/or the embodiments thereof, the pharmaceutical composition comprises from about 0.1 wt% to about 15 wt% of a poloxamer having the structure of Formula I,

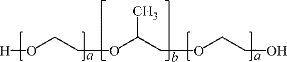

wherein the pH of the composition is between 4 to about 7.5 and wherein a is an integer from 50 to 120, and b is an integer from 15 to 40. In an embodiment, a is an integer from 60 to 90 and b is an integer from 25 to 35. In an embodiment, a=80 and b=27 (poloxamer 188). In an embodiment of the method, the pharmaceutical composition comprises from about 0.1 wt% to about 5 wt% of poloxamer 188. In an embodiment of the method, the pharmaceutical composition further comprises a saccharide. In an embodiment of the method, the pharmaceutical composition comprises from about 1 wt% to about 20 wt% of the saccharide. In an embodiment of the method, the pharmaceutical composition comprises from about 2 wt% to about 10 wt% of a saccharide. In an embodiment of the method, the pharmaceutical composition comprises a saccharide selected from a polyol, disaccharide and polysaccharide. In an embodiment, the saccharide is mannitol, lactose, or both mannitol and lactose. In an embodiment, the saccharide is D-mannitol.

In an embodiment of the method of any of the above-delineated aspects and/or the embodiments thereof, the pharmaceutical composition includes from 5 to 15 mg/ml of INS-HC1, from about 0.1 to about 5 wt% of poloxamer 188, and from about 1 to about 10 wt% of mannitol. In an embodiment of the method of any of the above-delineated aspects and/or the embodiments thereof, the pharmaceutical composition includes from 8 to 12 mg/ml of INS-HCI, from about 0.3 to about 1 wt% of poloxamer 188, and from about 3 to about 6 wt% of mannitol.

In an embodiment of the method of any of the above-delineated aspects and/or the embodiments thereof, the pharmaceutical composition is administered subcutaneously, intravenously, or intramuscularly. In an embodiment, the pharmaceutical composition is administered subcutaneously. In an embodiment of the method of any of the above-delineated aspects and/or the embodiments thereof, from 5 to 100 mg/day of INS-HCl is administered to the subject. In an embodiment of the method of any of the above-delineated aspects and/or the embodiments thereof, from 10 to 60 mg/day of INS-HCl is administered to the subject. In an embodiment of the method of any of the above-delineated aspects and/or the embodiments thereof, from 10 to 30 mg/day of INS-HCl is administered to the subject. In an embodiment of the method of any of the above-delineated aspects and/or the embodiments thereof, the method further involves administering an additional active agent to the subject. In an embodiment, the additional active agent is an anti-viral active agent.

In an embodiment of the above-delineated pharmaceutical compositions for use and/or embodiments thereof, the disease is treated in a mammal. In an embodiment of the above-delineated pharmaceutical compositions for use and/or embodiments thereof, the disease is treated in a human.

In an embodiment of the above-delineated methods and/or embodiments thereof, the subject is a mammal. In an embodiment of the above-delineated methods and/or embodiments thereof, the subject is a human.

In another aspect of the invention, a method of inhibiting coronavirus or influenza virus infection in a cell is provided, in which the method involves contacting a cell containing a coronavirus or an influenza virus with a peptide comprising the amino acid sequence WTAVQMAVFIHNFKRK (SEQ ID NO: 1, INS). In an embodiment, the coronavirus is a human coronavirus. In an embodiment, the human coronavirus is one or more of MERS-CoV, SARS-CoV and SARS-CoV-2 viruses. In an embodiment, the virus is influenza virus. In an embodiment, the influenza virus is influenza A virus.

In an embodiment of the method of inhibiting of the above-delineated aspects, the cell is a mammalian cell or is derived from a mammalian subject. In an embodiment of the methods of inhibiting of the above-delineated aspects, the cell is a human cell or is derived from a human subject. In an embodiment of the above-delineated methods, the cell is in vitro, in vivo, or ex vivo.

In an embodiment of the methods of inhibiting of the above-delineated aspects and/or embodiments thereof, the peptide is formulated in a composition. In an embodiment, the composition comprises a pharmaceutically acceptable carrier, diluent, or excipient. In an embodiment of the methods of inhibiting of the above-delineated aspects and/or embodiments thereof, the composition comprises from about 0.1 to about 30 mg/ml of hydrochloride salt of the peptide (INS-HCI). In an embodiment of the methods of inhibiting of the above-delineated aspects and/or embodiments thereof, the composition comprises from about 0.1 wt% to about 15 wt% of a poloxamer having the structure of Formula I,

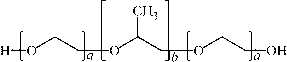

wherein the pH of the composition is between 4 to about 7.5 and wherein a is an integer from 50 to 120, and b is an integer from 15 to 40. In an embodiment, a is an integer from 60 to 90 and b is an integer from 25 to 35. In an embodiment, a=80 and b=27 (poloxamer 188). In an embodiment of the methods of inhibiting of the above-delineated aspects and/or embodiments thereof, the composition comprises from about 0.1 wt% to about 5 wt% of poloxamer 188. In an embodiment of the methods of inhibiting of the above-delineated aspects and/or embodiments thereof, the composition further comprises a saccharide. In an embodiment, the composition comprises from about 1 wt% to about 20 wt% of the saccharide. In an embodiment, the composition comprises from about 2 wt% to about 10 wt% of a saccharide. In an embodiment, the saccharide is selected from a polyol, disaccharide and polysaccharide. In an embodiment, the saccharide is selected from mannitol, lactose, or both mannitol and lactose. In an embodiment, the saccharide is D-mannitol. In an embodiment of the methods of inhibiting of the above-delineated aspects and/or embodiments thereof, the composition comprises from 5 to 15 mg/ml of INS-HCI, from about 0.1 to about 5 wt% of poloxamer 188, and from about 1 to about 10 wt% of mannitol. In an embodiment of the methods of inhibiting of the above-delineated aspects and/or embodiments thereof, the composition comprises from 8 to 12 mg/ml of INS-HCI, from about 0.3 to about 1 wt% of poloxamer 188, and from about 3 to about 6 wt% of mannitol.

Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person of ordinary skill in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “active agent” refers to an agent that has biological activity, pharmacologic effects and/or therapeutic utility. In some embodiments, an active agent is a peptide comprising or consisting of WTAVQMAVFIHNFKRK (SEQ ID NO: 1, INS).

By “agent” is meant any small molecule chemical compound, nucleic acid molecule, polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. ”

By “analog” is meant a molecule that is not identical, but has analogous functional or structural features. For example, a peptide analog retains the biological activity of a reference peptide, while having certain biochemical modifications that enhance the analog’s function relative to the reference. Such biochemical modifications could increase the analog’s protease resistance, membrane permeability, or half-life, without altering, for example, the biological function of the reference peptide. An analog may include an unnatural amino acid.

The terms “buffer,” “buffering system,” and/or “buffer solution” refer to compounds which reduce the change of pH upon addition of small amounts of acid or base, or upon dilution. The term “buffering agent” refers to a weak acid or weak base in a buffer solution.

The term “solution” as used herein refers to a clear, homogeneous liquid dosage form that contains one or more chemical substances dissolved in a solvent or in a mixture of mutually miscible solvents.

As used herein, the term “clear solution” refers to essentially transparent solutions devoid of particles above 100 nm. Alternatively, the term “clear solution” refers to essentially transparent solutions devoid of particles above 50 nm. Alternatively, the term “clear solution” refers to essentially transparent solutions devoid of particles above 40 nm.

The term “coadministration” or “coadministering” encompasses administration of a first and second agent in an essentially simultaneous manner, such as in a single dosage form, e.g., a capsule or tablet having a fixed ratio of first and second amounts, or in multiple dosage forms for each. The agents can be administered in a sequential manner in either order. When coadministration involves the separate administration of each agent, the agents are administered sufficiently close in time to have the desired effect (e.g., complex formation).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include COVID-19, influenza, and other viral infections.

By “effective amount” is meant the amount of an agent required to ameliorate the symptoms of a disease relative to an untreated patient. In some embodiments, an effective amount is an amount of an agent required to suppress, reduce, or eliminate a viral infection. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increases” is meant a positive alteration of at least 10%, 25%, 50%, 75%, or 100%.

The term “influenza virus” refers to a segmented negative-strand RNA virus that belongs to the Orthomyxoviridae family of viruses. There are three types of Influenza viruses: A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as, without limitation, H5N1, H5N2, H5N6, H5N8, H7N9, H9N2, H1N1, H1N2, H2N1, H2N2, H2N3, H7N3, H7N7, H3N2, H3N1, and related viruses, cause systemic infections in poultry in which mortality may reach 100%. H5N1 is also referred to as “avian influenza.” In an embodiment, the virus is influenza A virus, which causes influenza disease in humans.

The terms “intermittent” or “intermittently” as used herein means stopping and starting at either regular or irregular intervals. For example, intermittent administration can be administration in one to six days per week or it may mean administration in cycles (e.g. daily administration for two to eight consecutive weeks, then a rest period with no administration for up to one week) or it may mean administration on alternate days.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” nucleic acid or protein is sufficiently free of other materials such that any impurities do not materially affect the biological properties of the nucleic acid or protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, “obtaining” as in “obtaining an agent” includes synthesizing, purchasing, or otherwise acquiring the agent.

The term “pharmaceutical composition” as used herein refers to a composition comprising at least one active agent as disclosed herein optionally formulated together with one or more pharmaceutically acceptable carriers. Formulation of the pharmaceutical composition may be adjusted according to their intended use and administration route. In particular, the pharmaceutical composition may be formulated using a method known in the art so as to provide rapid, continuous or delayed release of the active ingredient after administration to mammals. In some embodiments, the pharmaceutical composition is formulated for a parenteral administration.

The phrase “pharmaceutically acceptable carrier” is recognized in the art and includes a pharmaceutically acceptable material, composition or vehicle, suitable for administering compounds of the present disclosure to a subject.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

The term “polyol” refers to compounds containing multiple hydroxyl groups and typically do not comprise other functional groups. Non-limiting examples of sugar alcohols such as sorbitol and mannitol. In some embodiments, the saccharide is mannitol. In some embodiments, the mannitol is D-mannitol.

The term “poloxamer” as used herein refer to non-ionic poly (ethylene oxide) (PEO)-poly (propylene oxide) (PPO) copolymers.

By “reduces” is meant a negative alteration of at least 10%, 25%, 50%, 75%, or 100%.

By “reference” is meant a standard or control condition. As used herein, “changed as compared to a reference” sample or subject or the like is understood as having a level that is statistically different than a sample from a normal, healthy, untreated, or reference sample. Reference samples include, for example, cells in culture, one or more laboratory test animals, or one or more human subjects. Methods to select and test reference samples are within the ability of those in the art. Determination of statistical significance is within the ability of those skilled in the art, e.g., the number of standard deviations from the mean that constitute a positive result. In one embodiment, the response of a subject having a disease (e.g., COVID-19, influenza virus infection) treated with an agent is compared to a reference, which would include the response of an untreated control subject, a healthy or normal (non-diseased) subject, or the disease state of the subject prior to treatment.

By “substantially identical” is meant a polypeptide or nucleic acid molecule exhibiting at least 85% identity to a reference amino acid sequence (for example, any one of the amino acid sequences described herein) or nucleic acid sequence (for example, any one of the nucleic acid sequences described herein). In some embodiments, the polypeptide or nucleic acid molecule is at least 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e′¹⁰⁰ indicating a closely related sequence.

Nucleic acid molecules useful in the methods of the invention include any nucleic acid molecule that encodes a polypeptide of the invention or a fragment thereof. In one embodiment, the nucleic acid molecule encodes a peptide of SEQ ID NO. 1 or 2. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “stable” as used herein refer to a composition, for example a solution, in which at least 80% of the peptide remains dissolved for at least 24 hours at room temperature (about 25° C.).

By “subject” is meant a mammal, including, but not limited to, a human or non-human mammal, such as a bovine, equine, canine, ovine, rodent, or feline.

The terms “substantially devoid”, “essentially devoid”, “devoid”, “does not include” and “does not comprise” may be used interchangeably and refer to composition that does not include, contain or comprise a particular compound or particles, e.g., the composition comprises less than 0.1 %, less than 0.01 %, or less than 0.001 % of the such compounds or particles. In some embodiments, the term devoid contemplate composition comprising traces of the devoid compounds or particles.

The term “sequential manner” refers to an administration of two compounds at different times, and optionally in different modes of administration. The agents can be administered in a sequential manner in either order.

The terms “substantially simultaneous manner” refers to administration of two compounds with only a short time interval between them. In some embodiments, the time interval is in the range of from 0.5 to 60 minutes.

The term “salts” refers to the relatively non-toxic, inorganic and organic acid addition salts of compounds of the present disclosure.

The term “treating” a condition or patient refers to taking steps to obtain beneficial or desired results, including clinical results. Beneficial or desired clinical results include, but are not limited to, ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

A “vector” refers to a nucleic acid (polynucleotide) molecule into which a heterologous nucleic acid can be inserted without disrupting the ability of the vector to replicate in and/or integrate into a host cell. In an embodiment, the foreign nucleic acid encodes a protein or peptide, such as the INS (GAMMORA™ or Codivir peptide) described herein. A vector can include nucleic acid sequences that permit it to replicate in a host cell, such as an origin of replication. An insertional vector can insert itself into a host nucleic acid. A vector can also include one or more selectable marker genes and other genetic elements. An expression vector is a vector that contains the necessary regulatory sequences to allow transcription and translation of an inserted gene or genes in a host cell. In some embodiments, the vector encodes GAMMORA™ or Codivir peptide. In some embodiments, the vector is an expression vector that allows the peptide to be expressed in a cell via transfection, transduction or infection. In embodiments, the vector is a viral vector or a plasmid vector which may be engineered to contain a nucleotide sequence which encodes the GAMMORA™ or Codivir peptide, using techniques and protocols that are well-known and used in the art.

As used herein the term “wt%” refers to % weight/weight as well known in the art. In any one of the embodiments of the present invention the term “wt%” may be substituted by the term “% wt/v”. The term “% wt/v” refers to percent weight/volume as well known in the art.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about”, when referring to a measurable value such as an amount, a temporal duration, and the like, is meant to encompass variations of +/-10%, or +/-5%, +/-1%, or even +/-0.1% from the specified value.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C present graphs showing the percentage of inhibition of SARS-CoV2 infection (black line) in Vero cells at different peptide (or control) concentrations. Cytotoxicity is displayed in light gray, as described in Example 2. The graphs in FIGS. 1A and 1B show the percentage of inhibition of SARS-CoV2 infection (black line) at different peptide (Codivir) concentrations. The x axes show compound (Codivir) dilutions (uM). FIG. 1C shows a positive control using Remdesivir. The curves represent the best fit of the logarithm of compound dilution vs. the normalized percentage of inhibition (variable slope).

FIG. 2 illustrates a representative image of untreated SARS-CoV2 infected cells (0.05% Acetic Acid). Infected cells show staining for the virus spike protein. Nuclei staining: total cells.

FIG. 3 illustrates a representative image of untreated uninfected cells (0.05% Acetic Acid). Nuclei staining of total cells.

FIG. 4 illustrates a representative assay plate showing the effect of the INS peptide on SARS-CoV2 infected cells. Infected untreated cells are brightly stained for the virus spike protein. All cells were stained with a nuclear strain. Infected cells treated with increasing concentrations of the Codivir peptide show a corresponding decrease in staining for the viral spike protein.

FIGS. 5A-5C present graphs showing the percentage of inhibition of Influenza A infection (black line) at different peptide (or control) concentrations in MDCK-II cells. Cytotoxicity is displayed in light gray, as described in Example 3. The graphs in FIGS. 5A and 5B show the percentage of inhibition of IAV infection (black line) at different peptide (Codivir) concentrations. The x axes show compound dilutions (_{µ}M). FIG. 5C shows a positive control using Niclosamide. The curves represent the best fit of the logarithm of compound (Codivir) dilution vs. the normalized percentage of inhibition (variable slope). Cytotoxicity is displayed in light gray.

FIG. 6 illustrates a representative image of untreated Influenza A-virus infected MDCK-II cells (0.05% Acetic Acid). Bright staining is observed in infected cells stained for the virus nucleoprotein. The total number of cells is visualized using nuclei staining.

FIG. 7 illustrates a representative image of untreated uninfected MDCK-II cells (0.05% Acetic Acid); nuclei staining: total cells.

FIG. 8 illustrates a representative assay plate showing the effect of the INS peptide on Influenza A infected cells. Cells in the wells were stained for the virus nucleoprotein (denoted by lighter gray in the image); nuclei staining: total cells.

### DETAILED DESCRIPTION OF THE INVENTION

The invention provides compositions and methods for treating a viral disease or infection.

The invention is based, at least in part, on the discovery that Codivir peptides disclosed herein (e.g. SEQ ID NO: 1; SEQ ID NO: 2) are useful for treating diseases associated with coronavirus or influenza virus infections, and that the same peptides inhibit infections by the viruses. More specifically, it was found that after 9 days of treatment, all 15 patients treated with a therapeutic composition comprising a Codivir peptide comprising WTAVQMAVFIHNFKRK (SEQ ID NO: 1) were discharged from the intensive care unit (ICU) to a general ward with almost full recovery. Four of the 15 patients treated were SARS-CoV-2-negative and completely recovered. In the control group, 14 out of 15 patients died. Accordingly, the invention provides methods for preventing or treating a viral infection (e.g., influenza, Covid) with peptide compositions described herein.

### Viral Infections

As detailed herein below, Codivir peptides described herein (e.g., SEQ ID NO: 1: WTAVQMAVFIHNFKRK; SEQ ID NO: 2: TAVQMAVFIHNFKRK) were effective in inhibiting viral infections associated with corona viruses and influenza.

Severe acute respiratory syndrome coronavirus 2 (SARS-CoV-2), formerly known as the 2019 novel coronavirus (2019-nCoV), is a positive-sense single-stranded RNA virus. It is contagious among humans and associated with coronavirus disease 2019 (COVID-19). SARS-CoV-2 has strong genetic similarity to known bat coronaviruses, making a zoonotic origin in bats likely, although an intermediate reservoir such as a pangolin is thought to be involved. From a taxonomic perspective, SARS-CoV-2 is classified as a strain of the species severe acute respiratory syndrome-related coronavirus. SARS-CoV-2 is the cause of the ongoing 2019-20 coronavirus outbreak, a Public Health Emergency of International Concern that originated in Wuhan, China.

In some embodiments, the coronavirus is a human coronavirus selected from the group consisting of Human coronavirus 229E (HCoV-229E), Human coronavirus OC43 (HCoV-OC43), Severe acute respiratory syndrome coronavirus (SARS-CoV), Human coronavirus NL63 (HCoV-NL63, New Haven coronavirus), Human coronavirus HKU1, Middle East respiratory syndrome-related coronavirus (MERS-CoV), and Severe acute respiratory syndrome coronavirus 2 (SARS-CoV-2). In some embodiments, the human coronavirus is selected from MERS-CoV, SARS-CoV and SARS-CoV-2. Thus, In some embodiments, the present invention provides a pharmaceutical composition comprising a peptide INS or a pharmaceutically acceptable salt thereof, and a pharmaceutically acceptable carrier, for use in treatment of a disease caused by a human coronavirus selected from MERS-CoV, SARS-CoV and SARS-CoV-2. In some embodiments, the disease is COVID-19. Thus, in some embodiments, the disclosure provides pharmaceutical compositions for use in treating COVID-19.

In other embodiments, a composition comprising a peptide of SEQ ID NO. 1 or 2 is useful for treating infection with an influenza virus. There are four types of influenza viruses: A, B, C and D. Human influenza A and B viruses cause seasonal epidemics of disease. Influenza A viruses are the only influenza viruses known to cause global epidemics of flu disease. A pandemic can occur when a new and very different influenza A virus emerges that both infects people and has the ability to spread efficiently between people. Influenza type C infections generally cause mild illness. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include: A(H1N1) and A(H3N2). Influenza A subtypes can be further broken down into different genetic “clades” and “sub-clades.”

Based on the efficacy of the peptides of SEQ ID NO. 1 and 2 in treating corona virus and influenza virus infections, one of skill in the art would expect such peptides to be effective against a variety of viral pathogens. Examples of viruses that infect humans include, but are not limited to, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1 = internally transmitted; class 2 = parenterally transmitted (i.e. Hepatitis C); Norwalk and related viruses, and astroviruses).

### Methods for Characterizing a Viral Infection

Methods for detecting or monitoring a viral infection are known in the art, and described herein below. Methods for assaying infectivity include, but are not limited to plaque assays, which are used to count discrete “infectious centers,” endpoint dilution assays, where the results are reported as Tissue Culture Infectious Doses 50% (TCID50) where one TCID50 is the amount of sample that will infect 50% of the test units.

Chemical and/or physical methods of virus quantification include, but are not limited to direct visualization of virions by electron microscopy, hemagglutination (HA) assay, and serological assays (based on antigen-antibody interactions). Examples of serologic assays include enzyme-linked immunosorbent assays (ELISA), fluorescent-tagged antibody assays, and precipitation assays.

Other methods for assaying a viral infection include, but are not limited to, genome quantification by PCR, hemagglutination inhibition assay and high throughput sequencing.

In some embodiments, influenza A or B is detected using a rapid influenza test, digital immune assay, or rapid nucleic acid amplification test. Such tests may be conducted at a point of care.

In some embodiments, SARS-CoV-2 is diagnosed using nucleic acid testing (e.g., reverse transcription PCR, isothermal amplification, protein testing, and point of care testing). Diagnostic methods are known in the art and described, for example, by Udugama et al. (ACS Nano. 2020 Mar 30 : acsnano.0c02624), which is incorporated herein by reference in its entirety.

### Reporting the Status

Additional embodiments of the invention relate to the communication of assay results, characterization of disease, or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis is communicated to the subject as soon as possible after the diagnosis is obtained. The diagnosis may be communicated to the subject by the subject’s treating physician. Alternatively, the diagnosis may be sent to a subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Methods of Treatment

Compositions comprising peptides of SEQ ID NO. 1 or 2, and derivatives or analogs thereof, are useful for treating viral infections. In one therapeutic approach, s protein or peptide, such as the INS (GAMMORA™ or Codivir peptide) described herein is administered systemically. The dosage of the administered agent depends on a number of factors, including the size and health of the individual patient. For any particular subject, the specific dosage regimes should be adjusted over time according to the individual need and the professional judgement of the person administering or supervising the administration of the compositions.

In some embodiments, treating a viral infection or virus-associated disease (e.g., influenza, COVID-19) comprises alleviating at least one of the symptoms of the disease. Thus, in some embodiments, treating a disease comprises at least one of reducing temperature, improving oxygenation, improving respiratory parameters such as respiratory rate, reducing viral load, and obtaining negative test for a virus. In some embodiments, the virus comprises SARS-CoV-2, Influenza A, or any combination thereof.

### Pharmaceutical Compositions

The present invention contemplates pharmaceutical preparations comprising peptides described herein (e.g., SEQ ID NO. 1, 2) For therapeutic uses, the peptide compositions identified herein are administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington’s Pharmaceutical Sciences by E. W. Martin. The amount of the therapeutic agent to be administered varies depending upon the manner of administration, the age and body weight of the patient, and with the clinical symptoms of the pathogen infection

Embodiments of the present invention are directed to stable pharmaceutical compositions comprising for parenteral administration one or more peptides defined, at least in part, by the amino acid sequence TAVQMAVFIHNFKRK (SEQ ID NO: 2) and their use in treating viral infections and diseases. In one or more embodiments, the pharmaceutical composition is a stable pharmaceutical composition. In one or more embodiments, the pharmaceutical compositions are administered parenterally. In one or more embodiments, the viruses comprise at least one of an influenza virus, a coronavirus, or any combination thereof. Encouraging results have been observed in treating COVID-19 subjects with a composition comprising a peptide comprising SEQ ID NO:2.

In some embodiments, the invention provides a pharmaceutical composition comprising from about 0.1 mg/ml to about 30 mg/ml of a salt of a peptide of 15 to 30 amino acids that comprises the sequence TAVQMAVFIHNFKRK (SEQ ID NO: 2), or of a derivative, fragment, or analog thereof, and a pharmaceutically acceptable carrier, for use in treatment of a disease caused by a coronavirus. In some embodiments, the peptide comprises the amino acid sequence WTAVQMAVFIHNFKRK (SEQ ID NO: 1), or a derivative, or analog thereof. In some embodiments, the peptide consists of the amino acid sequence WTAVQMAVFIHNFKRK (SEQ ID NO: 1, also denoted hereinafter “INS”). In some embodiments, the peptide consists of the amino acid sequence TAVQMAVFIHNFKRK (SEQ ID NO: 2). In some embodiments, the peptide contained in the pharmaceutical compositions is in the form of halide salt. In specific some embodiments, the salt of the peptide is a hydrochloride salt. In specific some embodiments, the salt of the peptide is a hydrochloride salt (INS HCl). Thus, In some specific embodiments, the present invention provides a pharmaceutical composition comprising a peptide consisting of amino acid sequence WTAVQMAVFIHNFKRK (SEQ ID NO: 1, INS) or a pharmaceutically acceptable salt thereof, and a pharmaceutically acceptable carrier, for use in treatment of a disease caused by at least one of an influenza virus, a coronavirus, or any combination thereof. In some embodiments, the coronavirus is a human coronavirus. In other some embodiments, the human coronavirus is selected from MERS-CoV, SARS-CoV, and SARS-CoV-2. In certain some embodiments, the pharmaceutical composition of the present invention is for use in treating COVID-19. In some embodiments, the pharmaceutical composition of the present invention is for use in treating Influenza A. In any one of the above disclosed embodiments, the pharmaceutical composition comprises from about 0.1 to about 30 mg/ml of hydrochloride salt of at least one of the peptides WTAVQMAVFIHNFKRK (SEQ ID NO: 1), TAVQMAVFIHNFKRK (SEQ ID NO: 2), or any combination thereof.the peptide (INS-HCI). In some embodiments, the pharmaceutical composition comprises from about 0.1 wt% to about 15 wt% of a poloxamer having the structure of Formula I,

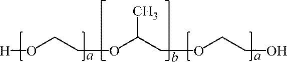

wherein the pH of the composition is between 4 to about 7.5 and wherein a is an integer from 50 to 120, and b is an integer from 15 to 40.

Thus, in some embodiments, the present invention provides a pharmaceutical composition comprising from about 0.1 to about 30 mg/ml of a salt of a peptide of 15 to 30 amino acids that comprises the sequence TAVQMAVFIHNFKRK (SEQ ID NO: 2), or of a derivative, fragment, or analog thereof, from about 0.05 wt% to about 15 wt% of a poloxamer, for use in treating of a disease caused by a coronavirus, wherein the poloxamer has the structure of Formula I,

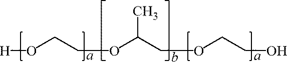

wherein a is an integer from 50 to 120, and b is an integer from 15 to 40, and a pharmaceutically acceptable carrier, wherein the pH of the composition is between about 4 to about 7.5. In some embodiments, the peptide is INS peptide. In specific some embodiments, the salt of the INS is a hydrochloride salt (INS HCl). In some embodiments, the coronavirus is a human coronavirus, such as MERS-CoV, SARS-CoV, and SARS-CoV-2.

In some embodiments, a in Formula I is an integer from 60 to 90 and b is an integer from 25 to 35. In some particular embodiments, a=80 and b=27 (poloxamer 188). In some embodiments, the pharmaceutical composition comprises from about 0.05 wt% to about 10 wt% of poloxamer 188. In some embodiments, the composition comprises from about 0.1 to about 5 wt% of poloxamer 188 and from about 5 mg/ml to about 15 mg/ml of the peptide INS HCl. In other some embodiments, the pharmaceutical composition comprises from about 0.1 wt% to about 2 wt% of poloxamer 188 and/or from about 8 mg/ml to about 12 mg/ml of the peptide INS HCl.

In some embodiments a pharmaceutical composition comprises a saccharide. In some embodiments, the saccharide is selected from a polyol, a disaccharide, and a polysaccharide. In some embodiments, the composition comprises from about 1 wt% to about 20 wt% of the saccharide. In other embodiments, the composition comprises from about 3 wt% to about 12 wt% of the saccharide. In some embodiments, the saccharide is a polyol (sugar alcohol). In some embodiments, the polyol is mannitol. In some embodiments, the saccharide is a disaccharide. In some embodiments, the disaccharide is lactose.

In some embodiments, the present invention provides a pharmaceutical composition comprising from about 0.1 wt% to about 10 wt% of poloxamer 188, from about 5 mg/ml to about 15 mg/ml of HCl salt of a peptide consisting of the amino acid sequence WTAVQMAVFIHNFKRK (SEQ ID NO: 1), and from about 1 wt% to about 10 wt% of mannitol.

In other some embodiments, the present invention provides a pharmaceutical composition comprising from about 2 wt% to about 8 wt% of mannitol, from about 0.5 wt% to about 2 wt% of poloxamer 188, and from about 8 mg/ml to about 12 mg/ml of INS HCl, for use in treating a disease or infection caused by at least one of an influenza virus, a coronavirus, or any combination thereof. In further embodiments, the pharmaceutical composition comprises from about 3 wt% to about 6 wt% lactose, from about 0.3 wt% to about 0.8 wt% of poloxamer 188, and from about 8 mg/ml to about 12 mg/ml of INS HCl. In some embodiments, lactose is used instead of mannitol.

In some embodiments, the pharmaceutical composition is a liquid or semi-liquid composition. In another some embodiments, the composition is a solution. In specific some embodiments, the solution is an aqueous solution. In yet other embodiments, the composition is a stable composition. In one some embodiments, the pharmaceutical composition is a parenteral composition, i.e. a composition suitable for parenteral administration such as intravenous, intramuscular, and subcutaneous administration.

In some embodiments, the composition is for use in as a parenteral administration. In some embodiments, the composition for use is administered via a route selected from: intramuscular (IM), intravenous (IV) and subcutaneous (SC). In one some embodiments, the composition is administered subcutaneously.

In some embodiments, the composition is for use that comprises administering to a subject from about 5 mg/day to about 100 mg/day of INS-HCI. In other some embodiments, the use comprises administering to a subject from about 10 mg/day to about 60 mg/day of INS-HCI. In some embodiments, the composition is administered to a subject every day. In some embodiments, the composition is administered to a subject 1, 2, 3 or 4 times a day.

In some embodiments, the composition comprises one or more additional active agents. In some embodiments, the additional active agent is an anti-viral agent, including but not limited to agents used to treat Ebola. Any additional active agent used to treat a coronavirus infection or influenza A infection may be used, in any treatment regimen, that includes the compositions of the present invention. In some embodiments, the additional agent is selected from the group consisting of chloroquine, hydroxychloroquine, and Remdesivir, or any combination thereof. The additional active agent may be administered before, simultaneously with, or after the compositions of the present invention.

In some embodiments, the present invention provides a method of treating a viral disease caused by a coronavirus or an influenza virus in a subject in need thereof, in which the method involves administering to the subject a pharmaceutical composition comprising from about 1 mg/ml to about 20 mg/ml of a salt of a peptide consisting of or comprising the amino acid sequence of SEQ ID NO: 1, and from 0.05 to 10 wt% of a poloxamer having a structure of Formula I,

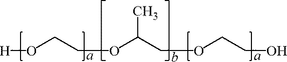

wherein the pH of the composition is between about 4 to about 7.5 and wherein a is an integer from 50 to 120, and b is an integer from 15 to 40. In some embodiments, the composition further comprises from about 1 wt% to about 10 wt% of a saccharide. In some embodiments, the saccharide comprises such as a polyol. In some embodiments, the present invention provides a method of treating COVID-19 in a subject in need thereof, in which the method involves administering to the subject a pharmaceutical composition comprising from about 1 mg/ml to about 20 mg/ml of INS-HCI, from about 0.05 wt% to about 10 wt% of poloxamer 188, and from about 1 wt % to about 10 wt% of mannitol. In some embodiments, the method comprises administering from about 10 mg/day to about 80 mg/day of INS-HCl to the subject. In some embodiments, the method comprises subcutaneous administration of the pharmaceutical composition subcutaneously.

In some embodiments, the present invention provides a pharmaceutical composition as described above herein per se. In some embodiments, the present invention provides a pharmaceutical composition comprising from about 1 mg/ml to about 20 mg/ml of a salt of a peptide consisting of the amino acid sequence of SEQ ID NO: 1, from 0.05 to 10 wt% of a poloxamer from 1 to 10 wt% of a polyol, wherein the poloxamer has a structure of Formula I,

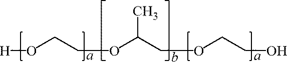

wherein the pH of the composition is between about 4 to about 7.5, and wherein a is an integer from 50 to 120, and b is an integer from 15 to 40. In some embodiments, the polyol is a mannitol. In some embodiments, wherein a=80, and b=27, and the poloxamer is poloxamer 188. In some embodiments, the INS is INS-HCI. In some embodiments, the present invention provides a pharmaceutical composition comprising from about 1 mg/ml to about 20 mg/ml of INS-HCI, from about 0.1 wt % to about 5 wt% of poloxamer 188, and from about 1 wt% to about 10 wt% of mannitol.

Pharmaceutical compositions of the invention to be used for therapeutic administration should be sterile. Sterility is readily accomplished by filtration through sterile filtration membranes (e.g., 0.2 µm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous solution comprising a peptide of SEQ ID NO. 1 or 2, or an analog or derivative thereof, such as an aqueous solution of such peptide and the resulting mixture can then be lyophilized. The infusion solution can be prepared by reconstituting the lyophilized material using sterile Water-for-Injection (WFI).

The polypeptides or analogs may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

Peptides of SEQ ID NO. 1 or 2, or an analog or derivative thereof of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris- hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, antimicrobials, inert gases, fluid and nutrient replenishers (i.e., Ringer’s dextrose), electrolyte replenishers, and the like, which can be present in conventional amounts.

The compositions, as described above, can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

### Formulation of Pharmaceutical Compositions

The administration of a peptide for the treatment of a viral infection may be by any suitable means that results in a concentration of the therapeutic that, combined with other components, is effective in ameliorating, reducing, or stabilizing a viral infection. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a pathogen infection by using carriers or chemical derivatives to deliver the therapeutic. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

With respect to a subject having a viral infection, an effective amount of a peptide described herein (e.g., SEQ ID NO: 1, 2) is sufficient to reduce a viral infection (e.g., influenza, Covid-19). Generally, doses of active polypeptide compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the peptide compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one embodiment, a composition of the invention comprising a peptide of SEQ ID NO. 1 or 2, or an analog or derivative thereof or a nucleic acid molecule encoding such peptide is administered by injection or infusion.

Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Parenteral Compositions

A pharmaceutical composition comprising a peptide (e.g., SEQ ID NO. 1, 2) is administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a viral infection, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable peptides are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer’s solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

The term “parenteral” includes subcutaneous, intrathecal, intravenous, intramuscular, intraperitoneal, or infusion.

Pharmaceutical compositions configured for parenteral administration include, but are not limited to, aqueous and non-aqueous sterile injectable solutions or suspensions, which may contain antioxidants, buffers, bacteriostats and solutes that render the compositions substantially isotonic with the blood of an intended subject or recipient. Such compounds do not decrease the stability or the solubility of the peptide provided in the composition.

In some embodiments, a is an integer from 55 to 115, from 60 to 110, from 65 to 105, from 70 to 100 or from 75 to 90. In some embodiments, b is an integer from 15 to 35, or from 20 to 30. In some embodiments, a is an integer from 60 to 90 and b is an integer from 25 to 35. In some embodiments, a=80 and b=27, and such a poloxamer is referred hereinafter as poloxamer 188. In some embodiments, poloxamer 188 has an average molecular weight between about 7600 kDa to about 9600 kDa. In some embodiments, poloxamer 188 has an average molecular weight of about 7800 kDa to about 9400 kDa, about 8000 kDa to about 9200 kDa, or about 8400 kDa to about 8800 kDa.

In some embodiments, the composition comprises from about 0.05 wt% to about 5 wt% poloxamer. In some embodiments, the composition comprises from about 0.1 wt% to about 5 wt% poloxamer. In some embodiments, the composition comprises from about 0.1 wt% to about 3 wt% poloxamer. In some embodiments, the composition comprises from about 0.3 wt% to about 1 wt% poloxamer. In some embodiments, the composition comprises from about 0.05 wt% to about 3 wt%, about 0.07 wt% to about 2.8 wt%, from about 0.08 wt% to about 2.6 wt%, from about 0.1 wt% to about 2.5 wt%, from about 0.2 wt% to about 2 wt%, from about 0.25 wt% to about 1.8 wt%, from about 0.3 wt% to about 1.5 wt%, from about 0.35 wt% to about 1.25 wt%, from about 0.4 wt% to about 1 wt%, from about 0.3 wt% to about 0.8 wt% or from about 0.2 wt% to about 0.6 wt% of poloxamer. In some embodiments, the composition comprises from about 0.1 wt% to 3 wt% poloxamer. In some embodiments, the composition comprises from about 5 wt% to about 10 wt% poloxamer. In some embodiments, the composition comprises from about 10 wt% to about 15 wt% poloxamer. In some embodiments, the composition comprises from about 15 wt% to about 20 wt% poloxamer. In some embodiments, the composition comprises from about 0.5 wt% to about 12 wt%, from about 0.6 wt% to about 10 wt%, from about 0.8 wt% to about 8 wt%, from about 1 wt% to about 6 wt%, from about 2 wt% to about 5 wt%, from about 3 wt% to about 4 wt% of poloxamer. In some embodiments, the pharmaceutical composition comprises from about 0.5 wt% to about 2 wt%, from about 0.6 wt% to about 1.8 wt%, from about 0.7 wt% to about 1.6 wt%, from about 0.8 wt% to about 1.4 wt%, or from about 0.9 wt% to about 1.2 wt% of the poloxamer. In some embodiments, the pharmaceutical composition comprises from about 0.6 wt% to about 1.2 wt% of the poloxamer. In some embodiments, the poloxamer is poloxamer 188. In some embodiments, the composition comprises from about 0.05 wt% to about 3 wt%, about 0.07 wt% to about 2.8 wt%, from about 0.08 wt% to about 2.6 wt%, from about 0.1 wt% to about 2.5 wt%, from about 0.2 wt% to about 2 wt%, from about 0.25 wt% to about 1.8 wt%, from about 0.3 wt% to about 1.5 wt%, from about 0.35 wt% to about 1.25 wt%, from about 0.4 wt% to about 1 wt%, from about 0.3 wt% to about 0.8 wt%, or from about 0.2 wt% to about 0.6 wt% of poloxamer 188.

In some embodiments, the composition comprises from about 0.1 mg/ml to about 30 mg/ml INS (SEQ ID NO: 1) salt. In some embodiments, the composition comprises from about 0.5 mg/ml to about 25 mg/ml, from about 1 mg/ml to about 20 mg/ml, from about 2.5 mg/ml to about 15 mg/ml, from about 5 mg/ml to about 12 mg/ml, from about 6 mg/ml to about 10 mg/ml, or from about 7 mg/ml to about 8 mg/ml INS salt. In some embodiments, the pharmaceutical composition comprises from about 0.5 wt% to about 12 wt%, from about 0.7 wt% to about 10 wt%, or from about 1 wt% to about 10 wt% INS as a salt. In some embodiments, the pharmaceutical composition comprises about 1, 2, 2.5, 3, 4, 5, 6, 7, 7.5, 8, 9, or 10 wt% of the peptide consisting of SEQ ID NO: 1. In any one of the disclosed embodiments, the peptide of the present invention is a salt. In some embodiments, the peptide consists of amino acid sequence SEQ ID NO: 1. In some embodiments, the composition comprises a trifluoroacetate salt of INS. In some embodiments, the composition comprises an acetate salt of INS. In some embodiments, the composition comprises a hydroxyl halide salt of INS. In some embodiments, the hydroxyl halide salt is selected from HCl, HBr and HI salt. In some embodiments, the composition comprises an HCl salt of INS, which may herein be denoted as “INS HCl”.

In some embodiments, the concentration or the amount of an INS salt relates to the peptide component of the salt. In some embodiments, a composition comprises 10 mg/ml of INS HCl. In some embodiments, a composition comprises 10 mg/ml of the peptide consisting of SEQ ID NO: 1. To calculate the content of the whole INS salt, the number should be multiplied by a correction factor of about 1.1, depending on the salt.

In some embodiments, the composition comprises from about 0.5 mg/ml to about 25 mg/ml, from about 1 mg/ml to about 20 mg/ml, from about 2.5 mg/ml to about 15 mg/ml, from about 5 mg/ml to about 12 mg/ml from about 6 mg/ml to about 10 mg/ml, or from about 7 mg/ml to about 8 mg/ml INS peptide as HCl salt. In some embodiments, the composition comprises from about 5 mg/ml to about 15 mg/ml, from about 5 mg/ml to about 20 mg/ml, from about 8 mg/ml to about 15 mg/ml, from about 8 mg/ml to about 12 mg/ml, or about 10 mg/ml INS-HC1. In some embodiments, the pharmaceutical composition comprises from about 0.5 wt% to about 12 wt%, from about 0.7 wt% to about 10 wt%, or from about 1 wt% to about 10 wt% of the peptide set forth in SEQ ID NO: 1 as HCl salt. In some embodiments, the pharmaceutical composition comprises 1, 2, 2.5, 3, 4, 5, 6, 7, 7.5, 8, 9, or 10 wt% of the peptide set forth in SEQ ID NO: 1 as HCl salt.

In some embodiments, the composition comprises from about 0.05 wt% to about 5 wt% of poloxamer 188 and from about 1 mg/ml to about 20 mg/ml, from about 2.5 mg/ml to about 15 mg/ml, from about 1 mg/ml to about 10 mg/ml, from about 2 mg/ml to about 10 mg/ml, or from about 5 mg/ml to about 10 mg/ml of the peptide INS HCl. In some embodiments, the composition comprises from about 0.1 wt% to about 3 wt% poloxamer 188 and from about 1 mg/ml to about 20 mg/ml, from about 2.5 mg/ml to about 15 mg/ml, or from about 5 mg/ml to about 10 mg/ml of the peptide INS HCl. In some embodiments, the composition comprises from about 0.3 wt% to about 2 wt% poloxamer 188 and from about 1 mg/ml to about 20 mg/ml, from about 2.5 mg/ml to about 15 mg/ml, from about 1 mg/ml to about 10 mg/ml, from about 2 mg/ml to about 10 mg/ml, or from about 5 mg/ml to about 10 mg/ml INS HCl. In some embodiments, the composition comprises from about 0.05 wt% to about 2 wt% poloxamer 188 and from about 1 mg/ml to about 20 mg/ml, from about 2.5 mg/ml to about 15 mg/ml, from about 1 mg/ml to about 10 mg/ml, from about 2 to about 10 mg/ml, or from about 5 mg/ml to about 10 mg/ml INS HCl.

In some embodiments, the composition comprises from about 0.05 wt% to about 5 wt% of poloxamer 188 and from about 1 mg/ml to about 15 mg/ml of the peptide INS HCl. In some embodiments, the composition comprises from about 0.3 wt% to about 2 wt% poloxamer 188 and from about 5 to about 15 mg/ml, from about 1 mg/ml to about 5 mg/ml, or from about 8 mg/ml to about 12 mg/ml of the peptide INS HCl.

In any one of the disclosed embodiments, the pharmaceutical composition has a pH of about 4 to about 7, about 4.5 to about 6.5, or from about 5 to about 6. In some embodiments, the pharmaceutical composition has a pH of about 4 to about 6.

In any one of the disclosed embodiments, the pharmaceutical composition further comprises a saccharide. In some embodiments, the saccharide comprises a monosaccharide, a disaccharide, a polyol, a polysaccharide, or any combination thereof. In some embodiments, the saccharide is a disaccharide. In some embodiments, the disaccharide comprises lactose, sucrose, lactulose, maltose, trehalose, cellobiose, chitobiose, or any combination thereof. In some embodiments, the disaccharide comprises lactose.

In some embodiments, the saccharide is a polysaccharide. In some embodiments, the polysaccharide is dextran.

In some embodiments, the saccharide is a polyol (sugar alcohol).

In some embodiments, the pharmaceutical composition comprises from about 1 wt% to about 20 wt% of the saccharide. In some embodiments, the composition comprises from about 2 wt% to about 18 wt%, from about 4 wt% to about 16 wt%, from about 6 wt% to about 14 wt%, or from about 8 to about 12 wt% of the saccharide. In some embodiments, the composition comprises from about 1 wt% to about 10 wt%, from about 2 wt% to about 8 wt%, from about 3 wt% to about 6 wt%, or from about 4 wt% to about 6 wt% of saccharide. In some embodiments, the saccharide is a disaccharide. In some embodiments, the saccharide is a polyol.

In some embodiments, the composition comprises from about 1 wt% to about 20 wt%, from about 2 wt% to about 18 wt%, from about 4 wt% to about 16 wt%, from about 6 wt% to about 14 wt%, or from about 8 wt% to about 12 wt% lactose. In some embodiments, the composition comprises from about 1 wt% to about 10 wt%, from about 2 wt% to about 8 wt%, from about 3 wt% to about 6 wt%, or from about 4 wt% to about 6 wt% lactose. In some embodiments, the composition comprises from about 6 wt% to about 12 wt% of lactose. In some embodiments, the lactose is anhydrous.

In some embodiments, the composition comprises from about 1 wt% to about 20 wt%, from about 2 wt% to about 18 wt%, from about 4 wt% to about 16 wt%, from about 6 wt% to about 14 wt%, or from about 8 wt% to about 12 wt% mannitol. In some embodiments, the composition comprises from about 1 wt% to about 10 wt%, from about 2 wt% to about 8 wt%, from about 3 wt% to about 6 wt%, or from about 4 wt% to about 6 wt% mannitol. In some embodiments, the pharmaceutical composition comprises from about 3 wt% to about 6 wt% mannitol.

In some embodiments, the pharmaceutical composition is devoid of Tween and/or ethanol.

In some embodiments, the pharmaceutical composition comprises from about 0.05 wt% to about 10 wt% of poloxamer 188, from about 1 mg/ml to about 20 mg/ml of HCl salt of the peptide consisting of amino acid sequence WTAVQMAVFIHNFKRK (SEQ ID NO: 1), and from about 1 wt% to about 12 wt% of a saccharide. In some embodiments, the saccharide is mannitol. In some embodiments, the saccharide is lactose.

In some embodiments, the poloxamer and the saccharides are used as pharmaceutically acceptable excipients.

In some embodiments, the pharmaceutical composition comprises from about 0.1 wt% to about 5 wt% poloxamer 188, from about 1 mg/ml to about 15 mg/ml or 1 mg/ml to about 12 mg/ml INS HC1, and from about 2 wt% to about 10 wt% of a saccharide. In some embodiments, the saccharide is mannitol. In some embodiments, the saccharide is lactose.

In some embodiments, the pharmaceutical composition comprises from about 0.2 wt% to about 2 wt% poloxamer 188, from about 5 mg/ml to about 15 mg/ml or about 5 mg/ml to about 12 mg/ml INS HC1, and from about 2 wt% to about 8 wt% mannitol. In some embodiments, the pH of the pharmaceutical composition is from 4.5 to 6.

In some embodiments, the pharmaceutical composition comprises from about 0.3 wt% to about 2 wt% poloxamer 188, from about 10 mg/ml to about 20 mg/ml of INS HC1, and from about 8 wt% to about 12 wt% lactose. In some embodiments, the pharmaceutical composition comprises from about 8 wt% to about 12 wt% lactose, from about 0.5 wt% to about 2 wt% poloxamer 188, and from about 1 mg/ml to about 5 mg/ml INS HCl. In some embodiments, the pharmaceutical composition comprises from about 8 wt% to about 12 wt% lactose, from about 0.8 wt% to about 2 wt% poloxamer 188, and from about 5 mg/ml to about 10 mg/ml INS HCl. In some embodiments, the pharmaceutical composition comprises from about 8 wt% to 12 wt% lactose, from about 0.8 wt% to about 2 wt% poloxamer 188, and from about 1 mg/ml to about 15 mg/ml INS HCl. In some embodiments, the pharmaceutical composition comprises from about 8 wt% to about 12 wt% lactose, from about 0.5 wt% to about 2 wt% poloxamer 188, and from about 1 mg/ml to about 12 mg/ml INS HCl. In some embodiments, the pH of the composition is from 4.5 to 6.

In some embodiments, the pharmaceutical composition comprises from about 0.5 wt% to about 10 wt% poloxamer 188, from about 5 mg/ml to about 15 mg/ml of HCl salt of the peptide consisting of amino acid sequence SEQ ID NO: 1, and from about 5 wt% to about 12 wt% of lactose. In some embodiments, the composition comprises from about 2.5 mg/ml to about 20 mg/ml INS HC1, 1% Poloxamer 188, and 9% Lactose anhydrous, wherein the composition has pH of about 5. In some embodiments, the composition comprises from about 2.5 mg/ml to about 20 mg/ml INS HC1, 0.8% Poloxamer 188, and 9.5% Lactose anhydrous, wherein the composition has pH of about 5. In some embodiments, the composition comprises 2.5, 5, 10, 15 or 20 mg/ml INS HCl. In some embodiments, the pH is from about 4.5 to about 5.5.

In some embodiments, the pharmaceutical composition comprises from about 0.1 wt% to about 4 wt% poloxamer 188, from about 5 mg/ml to about 20 mg/ml INS HC1, and from about 1 wt% to about 12 wt% mannitol. In a further embodiment, the pharmaceutical composition comprises from about 2 wt% to about 10 wt% mannitol, from about 0.2 wt% to about 3 wt% poloxamer 188, and from about 1 mg/ml to about 5 mg/ml INS HCl. In some embodiments, the pharmaceutical composition comprises from about 3 wt% to about 8 wt% mannitol, from about 0.3 wt% to about 3 wt% of poloxamer 188, from about 5 mg/ml to about 15 mg/ml INS HCl. In some embodiments, the pharmaceutical composition comprises from about 3.5 wt% to about 6 wt% lactose, from about 0.4 wt% to about 1 wt% of poloxamer 188, and from about 8 mg/ml to about 15 mg/ml INS HCl. In some embodiments, the pH of the composition is from 4.5 to 6.

As used herein the term “wt%” refers to % weight/weight as well known in the art. In any one of the embodiments of the present invention the term “wt%” may be substituted by the term “% wt/v”. The term “% wt/v” refers to percent weight/volume as well known in the art. In some embodiments, the pharmaceutical composition comprises from about 0.1 % wt/v to about 4% wt/v of poloxamer 188, from about 5 mg/ml to about 20 mg/ml of INS HC1, and from about 1% wt/v to about 12 % wt/v of mannitol. In some embodiments, the pharmaceutical composition comprises from about 2% wt/v to 10% wt/v mannitol, from about 0.2 wt/v to about 3% wt/v poloxamer 188, and from about 1 mg/ml to about 5 mg/ml INS HCl. In some embodiments, the pharmaceutical composition comprises from about 3% wt/v to 8% wt/v mannitol, from about 0.3% wt/v to about 3% wt/v poloxamer 188, from about 5 mg/ml to about 15 mg/ml INS HCl. In some embodiments, the pharmaceutical composition comprises from about 3.5% wt/v to 6 % wt/v lactose, from about 0.4% wt/v to about 1 % wt/v poloxamer 188, and from about 8 mg/ml to about 15 mg/ml INS HCl. In some embodiments, the pH of the composition is from 4.5 to 6.

In some embodiments, the pharmaceutical composition further comprises at least one buffer. In some embodiments, the buffer is an acetate buffer.

In some embodiments, the composition is a liquid composition. In some embodiments, the composition is a semi-liquid composition. In some embodiments, the liquid or semi-liquid composition is an aqueous composition.

In some embodiments, the composition is a solution.

In some embodiments, the composition is a clear solution.

In any one of the disclosed embodiments, the composition is a stable composition.

In some embodiments, at least 80 wt% of the peptide maintains dissolved for at least 2 days, at least 5 days, at least 7 days, or at least 14 days. In some embodiments, at least 85 wt% of the peptide remains dissolved for at least 2 days, at least 5 days, at least 7 days or at least 14 days. In some embodiments, at least 90 wt% or 95 wt% of the peptide remains dissolved for at least 2 days, at least 5 days, at least 7 days or at least 14 days.

In some embodiments, the composition is stable upon dilution with a phosphate buffered saline (PBS). In some embodiments, the composition remains a clear solution upon dilution with PBS. In some embodiments, the composition remains a stable for at least 1, 2, 3, 4, 5, 6, 7, 8, 9, 10, 11, 12, 13, or 14 days upon dilution in PBS. In some embodiments, the dilution is 1, 2, 3, 4, 5, 6, 7, 8, 9 or 10 times dilution. In some embodiments, no more than 10%, 5% or 3% of the INS peptide precipitates upon dilution of the pharmaceutical composition with PBS. In some embodiments, dilution is 10 times dilution.

In some embodiments, the pharmaceutical composition is a parenteral composition, i.e. the composition is formulated for parenteral administration. In embodiments, a parenteral is delivered by subcutaneous, intracutaneous, intravenous, intramuscular, intraarticular, intraarterial, intrasynovial, intrasternal, intrathecal, intralesional, intraperitoneal and intracranial injection, as well as various infusion techniques.

In some embodiments, parenteral administration is selected from intravenous, intramuscular, and subcutaneous administration. In some embodiments, the pharmaceutical composition is formulated for an administration route selected from intravenous, intramuscular, and subcutaneous administration. In some embodiments, the pharmaceutical composition is formulated for subcutaneous administration.

In some embodiments, the pharmaceutical composition according to any one of the disclosed embodiments is a lyophilized composition.

Compositions comprising a peptide of SEQ ID NO. 1 or 2, or an analog or derivative thereof can be added to a physiological fluid such as blood or synovial fluid. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising a peptide of SEQ ID NO. 1 or 2, or an analog or derivative thereof can contain multivalent metal ions, such as calcium ions, magnesium ions and/or manganese ions. Any multivalent metal ion that helps stabilize the peptide composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the peptides of SEQ ID NO. 1 or 2, or an analog or derivative thereof contained therein. Preferably, the non- aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000.

Suitable stable formulations can permit storage of the active agents in a frozen or an unfrozen liquid state. Stable liquid formulations can be stored at a temperature of at least -70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

Stable formulations comprising a peptide of SEQ ID NO. 1 or 2 are provided in a pharmaceutical composition comprising from about 0.1 mg/ml to about 30 mg/ml of a salt of a peptide comprising the amino acid sequence set forth in SEQ ID NO: 2 (TAVQMAVFIHNFKRK), from about 0.05 wt% to about 15 wt% of a poloxamer having the structure of Formula O,

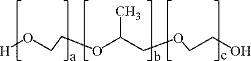

and a pharmaceutically acceptable carrier, wherein the pH of the composition is between about 4 to about 7.5 and wherein a and c are each independently an integer from 50 to 120, and b is an integer from 15 to 40, and the peptide consists of 15 to 40 amino acids.

In some embodiments, the peptide comprises the amino acid sequence set forth in SEQ ID NO: 1 (WTAVQMAVFIHNFKRK). In some embodiments, the peptide consists of 10 to 30 amino acids. In some embodiments, the peptide consists of the amino acid sequence WTAVQMAVFIHNFKRK (SEQ ID NO: 1), also denoted herein as “INS”. Thus, the term “peptide consisting of amino acid sequence SEQ ID NO: 1”, “peptide consisting of amino acid sequence WTAVQMAVFIHNFKRK” and “INS” are used herein interchangeably. In some embodiments, the peptide consists of the amino acid sequence set forth in SEQ ID NO: 2 (TAVQMAVFIHNFKRK).

In any one of the disclosed embodiments, any water-soluble salt of a peptide of the invention is contemplated. In some embodiments, the salt is trifluoroacetate salt. In some embodiments, the salt is acetate salt. In some embodiments, the salt is hydroxyl halide salt. In some embodiments, the hydroxyl halide salt is selected from HCl, HBr and HI salt.

In some embodiments, the water-soluble salt of the peptide is HCl. In some embodiments, the water-soluble salt of the INS peptide is HCl (denoted as INS-HCl). Thus, in some embodiments, the present invention provides a pharmaceutical composition comprising from about 0.1 mg/ml to about 30 mg/ml of INS-HCI.

In some embodiments, a and c in Formula O, are each independently an integer from 55 to 115, from 60 to 110, from 65 to 105, from 70 to 100 or from 75 to 90. In some embodiments, b is an integer from 15 to 35, or from 20 to 30. In some embodiments, a=c.

In some embodiments, the poloxamer has a structure of Formula I:

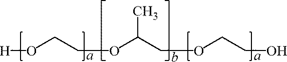

In some embodiments, the present invention provides a pharmaceutical composition comprising from about 0.1 mg/ml to about 30 mg/ml of a salt of a peptide consisting of 15 to 30 amino acid and comprising the amino acid sequence set forth in SEQ ID NO: 1 (WTAVQMAVFIHNFKRK), from about 0.05 wt% to about 15 wt% of a poloxamer having the structure of Formula I, and a pharmaceutically acceptable carrier, wherein the pH of the composition is between about 4 to about 7.5 and

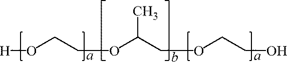

wherein a is an integer from 50 to 120, and b is an integer from 15 to 40.

In some embodiments, the present invention provides a pharmaceutical composition comprising from about 0.1 mg/ml to about 30 mg/ml of a salt of a peptide consisting of the amino acid sequence set forth in SEQ ID NO: 1, from about 0.05 wt% to about 15 wt% of a poloxamer having the structure of Formula I, and a pharmaceutically acceptable carrier, wherein the pH of the composition is between about 4 to about 7.5 and

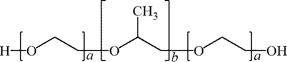

wherein a is an integer from 50 to 120, and b is an integer from 15 to 40.

A pharmaceutical composition refers to a composition comprising at least one active agent as disclosed herein optionally formulated together with one or more pharmaceutically acceptable carriers. Formulation of the pharmaceutical composition may be adjusted according to their intended use and administration route. In particular, the pharmaceutical composition may be formulated using a method known in the art so as to provide rapid, continuous or delayed release of the active ingredient after administration to mammals. According to one embodiment, the pharmaceutical composition is formulated for a parenteral administration.

Thus in some embodiments, the present invention provides a parenteral pharmaceutical composition comprising from about 0.1 mg/ml to about 30 mg/ml of a salt of a peptide consisting of amino acid sequence SEQ ID NO: 1, from about 0.05 wt% to about 15 wt% of a poloxamer having the structure of Formula I,

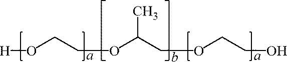

and a pharmaceutically acceptable carrier, wherein the pH of the composition is between about 4 to about 7.5, and wherein a is an integer from 50 to 120, and b is an integer from 15 to 40, and the pharmaceutical composition is configured for parenteral administration.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bio-erodible polymers such as polygalactin, poly-(isobutyl cyanoacrylate), poly(2-hydroxyethyl-L-glutam-nine) and poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

In some embodiments, a pharmaceutically acceptable carrier or pharmaceutically acceptable excipient includes any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (TWEEN® 20), polyoxyethylene sorbitan (TWEEN® 60), polyoxyethylene sorbitan monooleate (TWEEN® 80), sorbitan monopalmitate (SPAN® 40), sorbitan monostearate (SPAN® 60), sorbitan tristearate (SPAN® 65), glyceryl monooleate, sorbitan monooleate (SPAN® 80), polyoxyethylene esters (e.g., polyoxyethylene monostearate (MYRI® 45), polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether (BRIJ® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methyl cellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT® Plus, PHENONIPO, methylparaben, GERMALL® 115, GERMABEN® II, NEOLONEⓇ, KATHON®, and EUXYL®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer’s solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as CREMOPHORⓇ, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer’s solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

### Solid Dosage Forms

Formulations for oral use include tablets containing a peptide comprising SEQ ID NO. 1 or 2, or a derivative or analog thereof, in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active agent). The coating may be applied on the solid dosage form in a similar manner as that described in Encyclopedia of Pharmaceutical Technology, supra.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

In some embodiments, the present invention provides a solid pharmaceutical composition comprising a salt of a peptide comprising amino acid sequence TAVQMAVFIHNFKRK (SEQ ID NO: 2), a polyol, and a poloxamer having the structure of Formula O

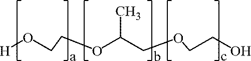

wherein a and c are each independently an integer from 50 to 120, and b is an integer from 15 to 40, and the peptide consists of 15 to 40 amino acids. In some embodiments, the weight ratio between the salt and the poloxamer is from about 10:1 to about 1:1500.

In some embodiments, the peptide comprises the amino acid sequence WTAVQMAVFIHNFKRK (SEQ ID NO: 1). In some embodiments, the peptide consists of amino acid sequence WTAVQMAVFIHNFKRK (SEQ ID NO: 1, INS). In some embodiments, the peptide consists of amino acid sequence TAVQMAVFIHNFKRK (SEQ ID NO: 2).

In some embodiments, a and c are each independently an integer from 55 to 115, from 60 to 110, from 65 to 105, from 70 to 100, or from 75 to 90. In some embodiments, b is an integer from 15 to 35, or from 20 to 30. In some embodiments, a=b.

In some embodiments, the poloxamer has the structure of Formula I:

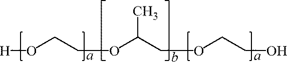

In some embodiments, the salt is hydroxyl halide salt. In some embodiments, the hydroxyl halide salt comprises at least one ofHCl, HBr, HI salt, or any combination thereof. In some embodiments, the composition comprises the peptide salt INS HCl.

In some embodiments, the present invention provides a solid pharmaceutical composition comprising a salt of a peptide consisting of amino acid sequence SEQ ID NO: 1 and a poloxamer having a structure of Formula I,

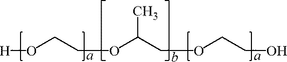

wherein a is an integer from 50 to 120, and b is an integer from 15 to 40. In some embodiments, a weight ratio between the INS salt and the poloxamer is from about 10:1 to about 1:1500. In some embodiments, a weight ratio between the salt and the poloxamer is from about 9:1 to about 1:1250, from about 8:1 to about 1:1100, from about 7:1 to about 1:800, from about 6:1 to about 1:600, from about 5:1 to about 1:400, from about 4:1 to about 1:200, from about 3:1 to about 1:100, from about 2:1 to about 1:50, or from about 1:1 to about 1:25. In some embodiments, a weight ratio between the salt and the poloxamer is from about 5:1 to about 1:50, from about 4:1 to about 1:40, from about 3:1 to about 1:30, from about 2:1 to about 1:20, or from about 1:1 to about 1:10.

In some embodiments, the poloxamer is as defined herein. In some embodiments, the poloxamer is poloxamer 188.

In some embodiments, the peptide salt is HCl salt of INS. In some embodiments, the present invention provides a solid pharmaceutical composition comprising an HCl salt of INS and a poloxamer 188, wherein the weight ratio between the salt and the poloxamer is from about 10:1 to about 1: 1000. In some embodiments, a weight ratio between INS HCl and poloxamer 188 is from about 5:1 to about 1:50, from about 4:1 to about 1:40, from about 3:1 to about 1:30, from about 2:1 to about 1:20, or from about 1:1 to about 1:10. In some embodiments, a weight ratio between the INS HCl and the poloxamer 188 is from about 2:1 to about 1:20.

In some embodiments, the polyol is as defined herein. In some embodiments, the polyol is mannitol. In some embodiments, the mannitol is D-mannitol.

In some embodiments, a weight ratio between the INS HCl and the polyol such as mannitol is from about 1:2000 to about 5:1.

In some embodiments, a weight ratio between the INS HCl and the mannitol is from about 1:1000 to about 4:1, from about 1:500 to about 3:1, from about 1:300 to about 2: 1, from about 1:100 to about 1:1, or from about 1:50 to about 1:10. In some embodiments, the weight ratio between the INS HCl and the lactose is from about 5:1 to about 1:30, from about 4:1 to about 1:24, from about 3:1 to about 1:20, from about 2:1 to about 1:15 or from about 1:1 to about 1:10.

In some embodiments, the present invention provides a solid pharmaceutical composition comprising the peptide salt INS HC1, poloxamer 188, and mannitol, wherein a weight ratio between INS HCl and poloxamer 188 is between about 3:1 to about 1:20, and a weight ratio between INS HCl and mannitol is between about 4:1 to about 1:24.

In some embodiments, the present invention provides a solid pharmaceutical composition comprising polyol and cumulatively comprising from about 0.9 wt% to about 70 wt% of an INS salt of and a poloxamer having a structure of Formula I,

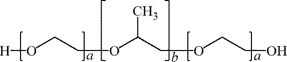

wherein a is an integer from 50 to 120, and b is an integer from 15 to 40. In some embodiments, a weight ratio between the INS peptide salt and the poloxamer is from about 10:1 to about 1: 1500. In some embodiments, a weight ratio between the peptide salt and the poloxamer is from about 9:1 to about 1:1250, from about 8:1 to about 1:1100, from about 7:1 to about 1:800, from about 6:1 to about 1:600, from about 5:1 to about 1:400, from about 4:1 to about 1:200, from about 3:1 to about 1:100 from about 2:1 to about 1:50 or from about 1:1 to about 1:25. In some embodiments, the weight ratio between the peptide salt and the poloxamer is from about 5:1 to about 1:50, from about 4:1 to about 1:40, from about 3:1 to about 1:30, from about 2:1 to about 1:20, or from about 1:1 to about 1:10.

In some embodiments, the poloxamer is as defined herein. In some embodiments, the poloxamer is poloxamer 188. In some embodiments, the salt is HCl salt of INS. In some embodiments, the present invention provides a solid pharmaceutical composition comprising from about 4.5 wt% to about 60 wt% of the peptide salt INS HCl and poloxamer 188. In some embodiments, the composition comprises from about 1.5 wt% to about 50 wt% of the peptide salt INS HC1.

In some embodiments, a weight ratio between the peptide INS HCl and polyol, such as mannitol, is from about 1:2000 to about 5:1.

In some embodiments, the present invention provides a solid pharmaceutical composition comprising from about 2 wt% to about 22 wt% INS HCl, from about 3.5 wt% to about 63 wt% poloxamer 188, and from about 30 wt% to about 92 wt% mannitol. In some embodiments, the solid pharmaceutical composition comprises from about 1.75 wt% to about 18.5 wt% INS HCl, from about 5 wt% to about 19.5 wt% poloxamer 188, and from about 95 wt% to about 89 wt% lactose. In some embodiments, the solid pharmaceutical composition comprises from about 2 wt% to about 20 wt% INS HCl, from about 8 wt% to about 9.5 wt% poloxamer 188, and from about 75 wt% to about 88 wt% mannitol.

In some embodiments, the solid pharmaceutical composition is a lyophilized composition.

In any one of the disclosed embodiments, the solid pharmaceutical composition of the present invention upon a reconstitution forms a liquid pharmaceutical composition of the present invention, e.g. a parenteral pharmaceutical composition.

In any one of the above embodiments, the solid pharmaceutical composition of the present invention upon a reconstitution comprises from about 0.1 to about 30 mg/ml of a salt of a peptide consisting of amino acid sequence WTAVQMAVFIHNFKRK (SEQ ID NO: 1), and from about 0.1 wt% to about 15 wt% of a poloxamer, and polyol, wherein the pH of the composition is between 4 to about 7.5 and the poloxamer has a structure of Formula I,

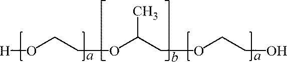

wherein a is an integer from 50 to 120, and b is an integer from 15 to 40.

In some embodiments, the present invention provides a method of treating a disease caused by a coronavirus or an influenza virus, the method comprising parenterally administering to a subject in need thereof an effective amount of the pharmaceutical composition comprising a peptide consisting of amino acid sequence WTAVQMAVFIHNFKRK (SEQ ID NO: 1) or a pharmaceutically acceptable salt thereof. In some embodiments, the present invention provides a method of treating a disease in a subject, the method comprising parenterally administering a therapeutically effective amount of a reconstituted solid pharmaceutical composition of the present invention. In some embodiments, the pharmaceutical composition of the present invention is administered subcutaneously. In some embodiments, the method comprises administering from about 10 to about 80 mg/day of INS-HCI.

In some embodiments, the present invention provides a method of treating a disease caused by a coronavirus or an influenza virus, the method comprising parenterally administering to a subject in need thereof an effective amount of the pharmaceutical composition comprising from about 0.1 mg/ml to about 30 mg/ml of a salt of a peptide consisting of 15 to 30 amino acids and comprising the amino acid sequence set forth in SEQ ID NO: 1 (WTAVQMAVFIHNFKRK ), from about 0.05 wt% to about 15 wt% of a poloxamer having the structure of Formula I, and a pharmaceutically acceptable carrier, wherein the pH of the composition is between 4 to about 7.5 and

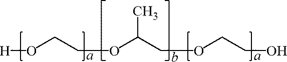

wherein a is an integer from 50 to 120, and b is an integer from 15 to 40.

In some embodiments, the pharmaceutical composition further comprises a saccharide, such as sugar alcohol. In some embodiments, the poloxamer is poloxamer 188.

In some embodiments, a method comprises administering, to a subject, a pharmaceutical composition comprising from about 0.05 wt% to about 12 wt% poloxamer 188, from about 1 mg/ml to about 20 mg/ml of an HC1 salt of the peptide consisting of amino acid sequence WTAVQMAVFIHNFKRK (SEQ ID NO: 1) and from about 1 wt% to about 10 wt% of a polyol. In some embodiments, the pharmaceutical composition comprises from about 0.1 wt% to about 4 wt% poloxamer 188, from about 5 mg/ml to about 20 mg/ml INS HC1, and from about 1 wt% to about 12 wt% mannitol. In some embodiments, the pharmaceutical composition comprises from about 2 wt% to about 10 wt% mannitol, from about 0.2 wt% to about 3 wt% poloxamer 188, and from about 1 mg/ml to about 5 mg/ml INS HC1. In some embodiments, the pharmaceutical composition comprises from about 3 wt% to about 8 wt% mannitol, from about 0.3 wt% to about 3 wt% poloxamer 188, from about 5 mg/ml to about 15 mg/ml INS HC1. In some embodiments, the pharmaceutical composition comprises from about 3.5 wt% to about 6 wt% lactose, from about 0.4 wt% to about 1 wt% poloxamer 188, and from about 8 mg/ml to about 15 mg/ml INS HC1. In some embodiments, the pH of the composition is from about 4.5 to about 6. In some embodiments, the pharmaceutical composition comprises from about 3% wt/v to 8% wt/v mannitol, from about 0.3% wt/v to about 3% wt/v poloxamer 188, from about 5 mg/ml to about 15 mg/ml INS HC1. In some embodiments, the pharmaceutical composition comprises from about 3.5% wt/v to about 6 % wt/v lactose, from about 0.4% wt/v to about 1 % wt/v poloxamer 188, and from about 8 mg/ml to about 15 mg/ml INS HC1. In some embodiments, the pH of the composition is from about 4.5 to about 6.

### Controlled Release Oral Dosage Forms

Controlled release compositions for oral use may, e.g., be constructed to release the active anti-viral therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Other Dosage Forms

Dosage forms for topical and/or transdermal administration of an agent (e.g., SEQ ID NO: 1 or SEQ ID NO: 2, and derivative or analogs thereof) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required.

Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Peptides described herein (e.g., SEQ ID NO. 1, SEQ ID NO: 2) can be incorporated into a variety of formulations for therapeutic use (e.g., by administration) or in the manufacture of a medicament by combining the agents with appropriate pharmaceutically acceptable carriers or diluents, and may be formulated into preparations in solid, semisolid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer’s solution, dextrose solution, and Hank’s solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents. toxicity adjusting agents, wetting agents and detergents.

Further examples of formulations that are suitable for various types of administration can be found in Remington’s Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977): 1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms.

Examples of particular esters include formates, acetates, propionates, butyrates, acrylates and ethylsuccinates.

Furthermore, the term “pharmaceutically acceptable prodrugs” as used herein refers to those prodrugs of the certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, i.e. the peptides of SEQ ID NO: 1 or SEQ ID NO: 2, in a biodegradable or bio-erodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers may be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Combination Therapies

In some embodiments, a pharmaceutical composition comprising a peptide (e.g., SEQ ID NO. 1, SEQ ID NO: 2) further comprises an additional active agent. In some embodiments, the additional active ingredient is an anti-viral agent. In some embodiments, the additional active agent is a protease inhibitor. In some embodiments, the protease inhibitor comprises darunavir, lopinavir, ritonavir or any combination thereof. In some embodiments, the additional active agent comprises atazanavir (ATV), amprenavir, fosamprenavir (APV), tipranavir (TPV), indinavir, saquinavir, lopinavir/ritonavir, prezista, nelfinavir (NFV), Remdesivir, azidothymidine (AZT), or any combination thereof. Nonlimiting examples of anti-viral compounds are compounds aimed at treating a viral infection caused by coronavirus or an influenza virus. In some embodiments, the additional anti-viral compounds are compounds useful for treating HIV.

In some embodiments, a use comprises co-administering a pharmaceutical composition with an additional active agent. In some embodiments, the additional active agent is an antiviral agent. In some embodiments, the antiviral agent is a protease inhibitor. In some embodiments, the protease inhibitor comprises at least one of lopinavir, darunavir, ritonavir or any combination thereof. In some embodiments, the active agent is an immunostimulant. In some embodiments, the active agent is an antibacterial agent.

### Administration of Pharmaceutical Compositions

The pharmaceutical compositions comprising a peptide described herein (e.g., SEQ ID NO. 1, SEQ ID NO: 2) are administered by any known method. The term “administering” or “administration of” a substance, a compound, an agent or a composition comprising any of them, to a subject can be carried out using one of a variety of methods known to those skilled in the art. For example, a compound or a composition comprising the agent can be administered, intravenously (IV), arterially, intradermally, intramuscularly (IM), intraperitoneally (IM), subcutaneously (SC), ocularly, sublingually, orally (by ingestion), intranasally (by inhalation), intraspinally, intracerebrally, intrathecally, rectally, and transdermally (by absorption, e.g., through a skin duct). A compound or a composition can also appropriately be introduced by rechargeable or biodegradable polymeric devices or other devices, e.g., patches and pumps, or formulations, which provide for the extended, slow or controlled release of the compound or agent. In some embodiments, the composition is administered intravenously. In some embodiments, the composition is administered intramuscularly. In some embodiments, the composition is administered subcutaneously.

The precise dose to be employed may vary depending on the route of administration and the progression of the disease or disorder, and should be decided according to the judgment of the practitioner and each patient’s circumstances. An exemplary 1 dosage of an INS peptide is in the range 0.01 to 5 mg/kg/dose. In some embodiments, the INS peptide is administered in the dosage of about 0.01 to 2, 0.05 to 1.5, 0.1 to 1.2, 0.2 to 1, 0.4 to 0.8, 0.3 to 0.7, or 0.4 to 0.6 mg/kg/dose. In some embodiments, the composition comprises from about 1 mg to about 30 mg INS HC1, from about 2.5 mg to about 20 mg, from about 5 mg to about 15 mg, or from about 5 mg to about 10 mg INS HC1. In some embodiments, the pharmaceutical composition is administered in the amount of about 10 to 50 mg/dose, about 15 to 40 mg/dose, or about 20 to 30 mg/dose. The composition may be administered as a single daily dose or in multiple doses. The administration schedule can be taken once-daily, twice-daily, thrice-daily, once-weekly, twice-weekly, thrice-weekly, once-monthly, twice-monthly, thrice-monthly, or any other administration schedule. In some embodiments, the pharmaceutical composition is administered twice a week. In some embodiments, the composition is administered in a dose of about 10 to 40 or about 15 to 30 mg twice a week. The administration may be continuous, i.e., every day, or intermittently.

In some embodiments, the method involves administering to a subject from about 5 mg/day to about 100 mg/day INS-HC1. In some embodiments, a use comprises administering to a subject from about 5 mg/day to about 80 mg/day, from about 10 mg/day to about 60 mg/day, or from about 15 mg/day to about 50 mg/day INS-HC1. In some embodiments, use of the pharmaceutical composition comprises administering to a subject from about 10 mg to about 30 mg, or from about 15 mg to about 25 mg INS-HC1 1, 2, 3 or 4 times a day.

In some embodiments, the present invention provides a pharmaceutical composition comprising a peptide salt according to any one of the disclosed embodiments.

### Delivery of Peptide Compositions

Pharmaceutical compositions of the present disclosure containing an agent described herein may be administered to an individual, such as a human individual, in need of treatment with an agent (e.g., SEQ ID NO: 1 or SEQ ID NO: 2, and derivatives or analogs thereof) in accord with known methods, such as oral administration, intravenous administration as a bolus or by continuous infusion over a period of time, by intramuscular, intraperitoneal, intracerebrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. et al. 1989, In: Toxicokinetics and New Drug Development, Yacobi et al., Eds, Pergamon Press, New York, pp. 42-46.

For in vivo administration of any of the agents of the present disclosure, normal dosage amounts may vary from about 10 ng/kg up to about 100 mg/kg of an individual’s and/or subject’s body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 µg/kg, followed by a weekly maintenance dose of about 100 µg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 µg/kg to about 2 mg/kg (such as about 3 µg/kg, about 10 µg/kg, about 30 µg/kg. about 100 µg/kg, about 300 µg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration.

Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In some embodiments of the present disclosure, a formulation as herein defined is administered to the subject by bolus administration.

A peptide described herein is administered to the subject in an amount sufficient to achieve a desired effect at a desired site (e.g., reduction of viral infection, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 µg/kg/day, at least 100 µg/kg/day, at least 250 µg/kg/day, at least 500 µg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 µg/kg/day, and less than 500 µg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 µg and 1 µg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., SEQ ID NO: 1 or SEQ ID NO: 2) described herein.

In certain embodiments, a dose described herein includes independently between 1 mg and 3 mg, inclusive, of an agent (e.g., SEQ ID NO: 1 or SEQ ID NO: 2 and derivatives or analogs thereof) described herein. In certain embodiments, a dose described herein includes independently between 3 mg and 10 mg, inclusive, of an agent (e.g., SEQ ID NO: 1 or SEQ ID NO: 2) described herein. In certain embodiments, a dose described herein includes independently between 10 mg and 30 mg, inclusive, of an agent (e.g., SEQ ID NO: 1 or SEQ ID NO: 2) described herein. In certain embodiments, a dose described herein includes independently between 30 mg and 100 mg, inclusive, of an agent (SEQ ID NO: 1 or SEQ ID NO: 2) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., SEQ ID NO: 1 or SEQ ID NO: 2) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., SEQ ID NO: 1 or SEQ ID NO: 2) described herein and an additional pharmaceutical agent shows a synergistic effect that is absent in a pharmaceutical composition including one of the agent and the additional pharmaceutical agent, but not both.

### Kits

The invention also provides kits for treatment or prevention of a viral disease or disorder (or symptoms) thereof, including a Covid-19, influenza, and symptoms thereof. In one embodiment, the kit includes an effective amount of a peptide described herein (e.g., SEQ ID NO. 1, 2, and derivatives or analogs thereof) in unit dosage form, together with instructions for administering the compound to a subject suffering from or susceptible to a viral disease or disorder or symptoms thereof. In other embodiments, the kit comprises a sterile container which contains the compound; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments.

In some embodiments, a kit comprises instructions that generally include information about the use of an agent described herein (e.g., SEQ ID NO. 1, SEQ ID NO: 2, and derivatives or analogs thereof) for treatment of a viral disease or disorder or symptoms thereof. In other embodiments, the instructions include at least one of the following: description of the compound; dosage schedule and administration for treatment of a viral disease or disorder or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

Having now generally described the invention, the same will be more readily understood through reference to the following examples, which are provided by way of illustration and are not intended to be limiting of the present invention.

### Example 1 - In Vivo Efficacy of INS Peptide in Treatment of COVID-19

The peptide denoted herein as INS, having the sequence WTAVQMAVFIHNFKRK (SEQ ID NO: 1) was prepared as described in WO 2010/041241, stored at -20° C., and used at a concentration of 2-15 mg/ml. The entirety of WO 2010/041241 is incorporated by reference herein.

A composition containing the INS peptide, e.g., as an HC1 salt, was prepared, and the pH was adjusted to between 4 to 6. In an embodiment, the composition comprises an aqueous pharmaceutical composition containing a poloxmer, e.g., POLOXAMER 188, and a saccharide, e.g., mannitol or D-mannitol, as shown in Table 1. POLOXAMER 188 (P188) (KOLLIPHOR^{®} P 188) was obtained from BASF, and D-Mannitol was obtained from Roquette.

TABLE 1

Composition Ingredient
INS peptide (as HC1 salt)
POLOXAMER 188
D-Mannitol
Hydrochloric acid (HCl), as needed
Sodium hydroxide (NaOH), as needed
Water (e.g., to 1 mL)

The safety, tolerability, and preliminary efficacy of INS peptide, SEQ ID NO: 1 (also herein referred to as GAMMORA™ or Codivir peptide) was measured by administering the composition described in Table 1 to patients infected with SARS-CoV-2 and comparing such patients to a control group. INS-HC1 peptide (GAMMORA™) 10 mg/ml (20 mg per administration) was administered once or twice daily via subcutaneous (SC) administration. The peptide was produced by Polypeptide USA. The Final Drug Product was formulated as specified in Table 1 and comprised 20 mg of INS-HC1 (2 ml of the composition). The Final Drug Product was produced and released under a sterile laminar hood by ZionMedical under Good Manufacturing Practice (GMP) conditions. Compositions comprising the GAMMORA™ peptide were administered to 15 patients positive for SARS-CoV-2 infection (coronavirus) with moderate to severe COVID-19 pneumonia.

Coronavirus infected patients that were enrolled in the study were allocated into 2 study groups in a 1:1 ratio. The patients of one group (Group 1) were administered the INS peptide subcutaneously (SC) at a dose level of 40 mg/day for 9 days, in addition to a standard of care antiviral treatment for Covid-19 at the time (such as Atripla comprising three different anti-HIV agents, Efavirenz, Emtricitabine and Tenofovir disoproxil fumarate, combined in one tablet). Treatment was administrated for a week at the medical center by trained and delegated staff members. The patients of the second group (Group 2) were a control group that received only Atripla.

All subjects were screened into the study and provided their consent prior to performing any study related activity. Eligible subjects per inclusion/exclusion criteria provided below were enrolled in the study. The treatment objectives of this study were to evaluate the safety and tolerability of the INS peptide (GAMMORA™) administered to patients infected with coronavirus and to evaluate preliminary anti-viral activity of INS peptide administered to patients positive to coronavirus.

Following allocation into one of the 2 study groups, patients received the first administration of the treatment. Each patient underwent a screening and a treatment for a period of 9 days, and a follow-up (FU) period of approximately 3 days.

Overall, 30 subjects were enrolled (15 subjects per study group) into the study. Evaluations after 3 days and/or 7 days were performed to decide if treatment should be continued. After the last treatment dose (at day 9), the patients were evaluated as follows; if improvement was observed, the treatment continued; if no improvement was observed, the treatment was stopped.

Safety related parameters and preliminary efficacy parameters were evaluated throughout the treatment and follow-up period.

Symptomatic treatment and antibiotics to prevent bacterial super infection were provided by investigators based on clinical judgment. The treatment endpoints included, without limitation, the following criteria: the number and proportion of treatment-related Adverse Events (AEs) in subjects administered with GAMMORA™ peptide compared to control group; oxygen levels; improvement in chest x-ray; percentage of mortality and ventilated patients; discharge from hospital; objective viral response: SARS-CoV-2 test - nasopharyngeal sample; liver and renal functions; a standardized clinical examination of the patient.

### Subject Monitoring

### Screening Day 0/Day 1 Visit

Screening of the patients was performed either on the first day of treatment or 1-2 days before that (referred as screening Day 0 Visit). Data collected included demographics (age, weight, height); concomitant medication, vital signs (respiratory rate, heart rate, temperature, blood pressure); length of stay at hospital, and mortality and occurrence of side effects. Oxygen levels were measured by the hours and days; full physical examination was performed, including examination of the following systems: skin, lymph nodes, head, eye, ear, nose and throat, respiratory, cardiovascular, gastrointestinal, neurologic and muscoskeletal; chest X-ray (every 2-3 days); blood tests; biochemistry: complete panel including renal function, liver function and CRP; hematology: white blood cells (WBC), hemoglobin, neutrophils, and platelets; coagulation:prothrombin time (PT), activated partial thromboplastin time (PTT) and International Normalized Ratio (INR), Inflammatory markers assessment.

The clinical laboratory assessments performed at screening visit (blood tests -biochemistry, hematology, coagulation) were defined as baseline assessments.

Without intending to be limiting, for inclusion in this particular study, subjects were required to meet the following criteria prior to enrollment: male or female patients aged >18 years; documented SARS-CoV-2 carriage in nasopharyngeal samples at admission; moderate to severe cases of COVID-19; adequate baseline organ function (hematologic, liver, renal and nutritional; patients or a legal guardian with the ability to understand and give written informed consent for receiving this treatment, including all evaluations and procedures as specified by this protocol; HIV patients taking Atripla or other HIV suppressing drug, other than an integrase inhibitor. Accordingly, all study subjects were Covid-19-positive. In an embodiment, HIV-positive patients taking Atripla were allowed for inclusion in the study, because Atripla was also used as a treatment for Covid-19 at the time of the study.

Without intending to be limiting, the exclusion criteria for the conditions of this particular study included the following: treatment with live attenuated vaccines in the last three weeks; women who are pregnant or lactating; women of child-bearing potential (WOCBP) and fertile men with a WOCBP partner, not using adequate birth control; patients with any severe hematologic abnormalities at baseline; patients with any sever serum chemistry abnormalities at baseline; patients with a significant cardiovascular disease or condition; patients with a history of active infection with hepatitis B virus (HBV) or hepatitis C virus (HCV); un-treated HIV patients; patients with any other life-threatening illness, significant organ system dysfunction, or clinically significant laboratory abnormality, which, in the opinion of the Investigator, would either compromise the patient’s safety or interfere with evaluation of the safety of the study drug; patients with a psychiatric disorder or altered mental status that would preclude understanding of the informed consent process and/or completion of the necessary studies; patients with the inability, in the opinion of the Investigator, to comply with the protocol requirements. It will be appreciated by the skilled practitioner that such inclusion and exclusion criteria are particular for the study described herein, and are not intended to limit the administration of the compound to a diversity of participants in other studies, regardless of the criteria selected for inclusion or exclusion in this particular study.

The safety assessments of this study included, without limitation, a complete physical examination (skin, lymph nodes, head, eye, ear, nose and throat [HEENT], respiratory, cardiovascular, gastrointestinal, neurologic and muscoskeletal), vital signs (heart rate, blood pressure, respiratory rate, and body temperature); clinical laboratory assessments (blood tests, e.g., biochemistry, hematology, coagulation, AE/SAE assessments and concomitant medication. The efficacy assessments in this particular study included, without limitation, respiratory rate, temperature, length of stay at hospital and mortality and occurrence of side effects; a SARS-CoV-2 test.

Eligible subjects who met all inclusion criteria and none of the exclusion criteria were invited to start the first treatment administration.

The eligible subjects that were enrolled into the study were divided in two groups in a 1:1 ratio. In total 15 subjects are enrolled to each one of the study groups in Table 2:

TABLE 2

| Group | No. of animals | Treatment (Total Daily Dose) |
|---|---|---|
| 1 | 15 | GAMMORA™ (40 mg/day for 9 days) |
| 2 | 15 | Control |

### Days 2-8 (Visits 2-8)

The following assessments were performed: AE/SAE assessments; Vital signs (respiratory rate, heart rate, temperature, blood pressure), long of stay at hospital and mortality and occurrence of side effects; Record of oxygen levels by the hours and days; Full physical examination including skin, lymph nodes, head, eye, ear, nose and throat, respiratory, cardiovascular, gastrointestinal, neurologic and muscoskeletal; Chest X-ray (every 2-3 days); Concomitant medication; Blood tests (taken at day 5±1day): Blood tests were taken at baseline, after 4 days and after 7 days of treatment as part of end of treatment assessments; Biochemistry: complete panel including renal function, liver function and CRP; Hematology - White Blood Cells (WBC), hemoglobin, neutrophils, and platelets; Coagulation - Prothrombin Time (PT), activated Partial Thromboplastin Time (PTT) and International Normalized Ratio (INR); Inflammatory markers assessment.

In cases of early treatment termination, the subjects were instructed to complete visit (visit 10) as soon as possible to termination date.

### Day 9 (Visit 9; End of Treatment Assessments)

End of treatment assessments were performed after last treatment dose with GAMMORA™ peptide. The following assessments was performed: AE/SAE assessments; Vital signs (respiratory rate, heart rate, temperature, blood pressure), long of stay at hospital and mortality and occurrence of side effects; Record of oxygen levels by the hours and days; Full physical examination including skin, lymph nodes, head, eye, ear, nose and throat, respiratory, cardiovascular, gastrointestinal, neurologic and muscoskeletal; Chest X-ray; Concomitant medication; Blood tests; Biochemistry: complete panel including renal function, liver function and CRP; Hematology - White Blood Cells (WBC), hemoglobin, neutrophils, and platelets; Coagulation - Prothrombin Time (PT), activated Partial Thromboplastin Time (PTT) and International Normalized Ratio (INR); Inflammatory markers assessment; Objective viral response; SARS-CoV-2 test - nasopharyngeal sample.

### Follow-Up (Day 12)

The following assessments were performed; AE/SAE assessments; Vital signs (respiratory rate, heart rate, temperature, blood pressure), long of stay at hospital and mortality and occurrence of side effects; Record of oxygen levels by the hours and days; Full physical examination including skin, lymph nodes, head, eye, ear, nose and throat, respiratory, cardiovascular, gastrointestinal, neurologic and muscoskeletal; Chest X-ray; Concomitant medication; Blood tests; Biochemistry: complete panel including renal function, liver function and CRP; Hematology - White Blood Cells (WBC), hemoglobin, neutrophils, and platelets; Coagulation - Prothrombin Time (PT), activated Partial Thromboplastin Time (PTT) and International Normalized Ratio (INR); Inflammatory markers assessment; Objective viral response; SARS-CoV-2 test -nasopharyngeal sample.

### Results

After nine days of treatment with a composition comprising the INS-HC1 peptide, all 15 patients in the treatment group were discharged from intensive care unit (ICU) to the general ward with almost full recovery and were standing and walking by themselves with no assistance. Four patients recovered completely and were negative for SARS-CoV-2 at the endo of the study.

In the control group, 14 out of 15 patients died. The remaining patients showed no improvement in their medical condition.

### Example 2: In Vitro Efficacy of INS Peptide Against SARS-CoV2

This study was performed to determine the cytotoxicity and antiviral activity of the synthetic INS peptide, Codivir, against SARS-CoV2, when administered at the same time as viral infection, or 2 hours (2 h) after infection.

### Materials and Methods

To determine the antiviral activity of Codivir, Vero cells were either infected with SARS-CoV2 for 2 h, and then treated with a three-fold serial dilution of peptide ranging from 75 µM to 0.05 µM, or treated with the same concentrations of peptide immediately before infection. Antiviral activity was determined at 24 h using an immunofluorescence-based assay. Cytotoxicity was determined using an MTT assay on uninfected cells treated with the same concentrations of peptide. The MTT assay is a colorimetric assay for assessing cell metabolic activity as known and used in the art. NAD(P)H-dependent cellular oxidoreductase enzymes under defined conditions reflect the number of viable cells present in an assayed sample of cells. (See, e.g., Stockert J.C. et al., April 2018, Acta Histochemica, 120(3):159-67; doi:10.1016/j.acthis.2018.02.005; PMID 29496266). Remdesivir was included as an assay control.

TABLE 3

Test article and stock preparation

| Compound ID | Molecular Weight | Weighted Mass | Diluent | Stock Concentration | Volume of diluent (µl) |
|---|---|---|---|---|---|
| Codivir | 1976.36 | 1.6 mg | 20% Acetic Acid | 5 mM | 160 |

Stored before reconstitution: -20° C.

Stored after reconstitution: -20° C.

### Controls

Uninfected untreated cells (with 0%, 0.05%, or 0.15% Acetic Acid)
Infected untreated cells (with 0%, 0.05%, or 0.15% Acetic Acid)
Positive control drug: Remdesivir (GS-5734)

### Test System

Cells: Vero E6, (VRS stock: ADp3)
Virus: SARS-CoV2/England/2/2020, from BEI Resources (VRS stock)
Readout: 1) High content immunofluorescence
2) Cell viability (cytotoxicity assays)
Standard Operating Procedures were employed for cell passaging, cell counting, infectivity and cytotoxicity assays; immunofluorescence staining; and the MTT assay.

### Key Reagents

Complete media: M199 medium (Gibco, 41150087) supplemented with 5% FBS (Gibco 10500064) and 1X p/s (Gibco 15070063)
Supplemented media: M199 medium (Gibco, 41150087) supplemented with 0.4% BSA (Gibco 15260037) and 1X p/s (Gibco 15070063)
Diluent: Acetic Acid (Analar Normapur, VWR 20104.334, Lot 13E030508); 20%: 2ml
Ac. Acid in 8ml water
Remdesivir (GS-5734), Selleckchem S8932, Lot: S893201

### Experimental Procedure

The antiviral activity of 8 dilutions of Codivir was explored following two modes of administration: at the same time as the virus (with infection), and 2 h after infection (post-infection) with SARS-CoV2. Virus and peptide were left on the cells for the entire duration of the experiment (24 h). The cytotoxicity of the same range of concentrations of Codivir was determined by MTT assay.

### Cell Plating

Cells were detached and counted following standard procedures. Count was recorded in a Cell Count Logbook. Cells were seeded in complete media at 8,000 cells/100µl/well in two plates: one for the cytotoxicity assay and one for the infectivity assay. After seeding, the plates were incubated at RT for 5 minutes for even distribution, and then at 37° C., 5% CO2 until the following day.

### Infection

The virus stock was diluted 10-fold to bring the concentration to 1x10 TCID50/ml. 2.8µl of diluted virus were transferred into 4.397.2µl of supplemented media (Multiplicity of Infection (MOI) 0.002). Media was removed from the cells and 50µl virus added in columns 1-10. 50µl of supplemented media without virus were added in columns 11-12 (uninfected control).

### Peptide Preparation

Two peptide stocks were prepared:
1) 150 µM stock
2) 25 µM stock

The initial 5 mg/ml (~ 5 mM, 20% Acetic Acid) Codivir stock was prepared as reported in Table 3.

160µl of water were added to bring the stock to 2.5 mM, 10% Acetic Acid
→ 78µl of 2.5 mM peptide were added to 1.222µl of supplemented media to reach the 150 µM concentration.
→13µl of 2.5 mM peptide were added to 1.287µl of supplemented media to reach the 25 µM concentration.

In order to preserve the same final concentration of Acetic Acid across the serial dilution (and no lower than 0.05%), three assay media were prepared:
1) supplemented media containing no Acetic Acid (“supplemented media”)
2) supplemented media containing the same concentration of Acetic Acid as the top dilutions of the 25 µM concentration (0.1%; “supplemented media + diluent 0.1%” → 12 ml + 12µl 100% Acetic Acid)
3) supplemented media containing the same concentration of Acetic Acid as the top dilution of the 150 µM concentration (0.3%; “supplemented media + diluent 0.3%” → 12 ml + 36µl 100% Acetic Acid)

120µl of “supplemented media + diluent 0.1%” were added in each row of columns 1-6 of a round-bottom 96 well plate, except rows A, B, C.

In row B, 120µl of “supplemented media” (no diluent) were added.

180µl of peptide at 150 µM were added to wells 1 to 6 of row A, and diluted 3-fold by transferring 60µl into row B.

180µl of peptide at 25 µM were added to wells 1 to 6 in row C, and serially diluted down (3-fold) by transferring 60µl to the next rows.

120 µl of “Supplemented media + diluent 0.1%” were added into columns 9 and 11, rows A to D.

120 µl of “supplemented media + diluent 0.3%” were added into columns 10 and 12, rows A to D.

120 µl of “supplemented media” were added into columns 9 to 12, rows E to H, to control for the effect of Acetic Acid on cells and virus.

2.6µl of a 10 mM stock or Remdesivir in DMSO were added to 697.4µl of supplemented media (40 µM).

180µl of 40 µM Remdesivir were added to row A of columns 7 and 8

120µl of supplemented media were added in the remaining rows.

60µl of 40 µM Remdesivir were moved down in a 3-fold dilution series.

### Peptide Treatment

Either immediately after infection, or 2 h after infection, 50µl of treatment from the dilution plate were transferred to the cells in each plate (infectivity and cytotoxicity). Remdesivir was added immediately after infection. 50 µl of media without diluent were added to the cytotoxicity plate instead of virus. Both plates were incubated at 37° C., 5% CO2 for 24 h.

### Fixation and Development

After 24 h, one plate was washed with PBS, fixed for 30 mins with 4% formaldehyde, washed again with PBS, and stored in PBS at 4° C. until staining. The cytotoxicity plate was treated with MTT to determine cell viability.

### Infectivity Readout

Cells were immunostained using standard protocols and techniques. Briefly, any residual formaldehyde was quenched with 50 mM ammonium chloride, after which cells were permeabilized (0.1% Triton X100) and stained with an antibody recognizing SARS—CoV2 spike protein (GeneTex GTX632604). The primary antibody was detected with an Alexa-488 conjugate secondary antibody (Life Technologies, A11001), and nuclei were stained with Hoechst. Images were acquired on an Opera Phenix high content confocal microscope (Perkin Elmer) using a 10X objective, and percentage infection calculated using Columbus software (infected cells/total cells x 100).

### Cytotoxicity Readout

Cytotoxicity was detected by MTT assay following standard protocols and techniques. Briefly, the MTT reagent (Sigma, M5655) was added to the cells for 2 h at 37° C., 5% CO2, after which the media was removed and the precipitate solubilized with a mixture of 1: 1 Isopropanol: DMSO for 20 minutes. The supernatant was transferred to a clean plate and signal read at 570 nm.

### Determination of EC50 Concentration - IF Assay

Normalized percentages of inhibition were calculated using the following formula:

$$\begin{matrix}N o r m a l i s e d \% i n h i b i t i o n \\ = 100 \times \\ \left(1 - \frac{\% I n f e c t i o n S a m p l e - \% I n f e c t i o n U n i n f e c t e d C o n t r o l}{\% I n f e c t i o n I n f e c t e d C o n t r o l - \% I n f e c t i o n U n i n f e c t e d C o n t r o l}\right)\end{matrix}$$

EC50 values were extrapolated from the curves representing the best fit (non-linear regression analysis, variable slope) of the logarithm of compound concentration vs. the normalized percentages of inhibition, using GraphPad Prism (version 9).

### Determination of TC50 Concentration

Percentages of cytotoxicity were calculated using the following formula:

$$\% c y t o t o x i c i t y = 100 - \left(100 \times \frac{A b s o r b a n c e S a m p l e}{A b s o r b a n c e U n t r e a t e d C o n t r o l}\right)$$

TC50 values were extrapolated from the curves representing the best fit (non-linear regression analysis, variable slope) of the logarithm of compound concentration vs. the normalized percentages of cytotoxicity, using GraphPad Prism (version 9).

### Summary of Results

Using an immunofluorescence-based assay at 24 h after infection, Codivir showed antiviral activity against SARS-CoV2 with an EC50 between 12.48 µM (when administered at the same time as infection) and 13.34 µM (when administered post infection), and a selectivity index of 3.48 or 3.26, respectively. The corresponding EC90 and SI90 values were 13.7 µM and 16.6 µM (EC90) and 10.5 and 8.6 (SI90). No significant cytotoxicity or antiviral activity was observed in the presence of vehicle (acetic acid) only.

### Results

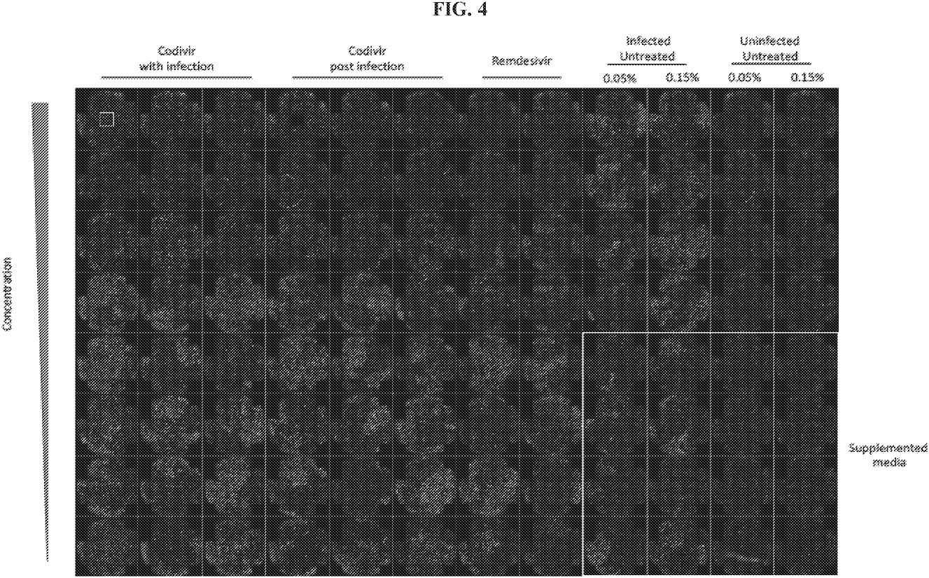

FIG. 2 is a representative image of untreated infected cells (0.05% Acetic Acid). White: infected cells, stained for the virus spike protein; gray: total cells, nuclei staining. FIG. 3 is a representative image of untreated uninfected cells (0.05% Acetic Acid). Gray: total cells, nuclei staining. FIG. 4 is an overview of the assay plate. White: infected cells, stained for the virus spike protein; gray: total cells, nuclei staining.

Table 4 displays the EC50, EC90, TC50, TC90 and Selectivity Index (SI) 50 and SI90 for Codivir and the Remdesivir control. Inhibition of SARS-CoV2 infection was observed in cells treated with Codivir, with EC50 of 12.48 µM (peptide administered at the same time as the virus) and 13.34 µM (peptide administered post infection). EC90 for the same experimental conditions was 13.7 µM and 16.6 µM, respectively. Some cytotoxicity was observed at the highest concentrations tested, with a TC50 value of 43.48 µM, and a TC90 value of 143.6 µM. The SI50 was between 3.48 and 3.26, and the SI90 between 10.5 and 8.6.

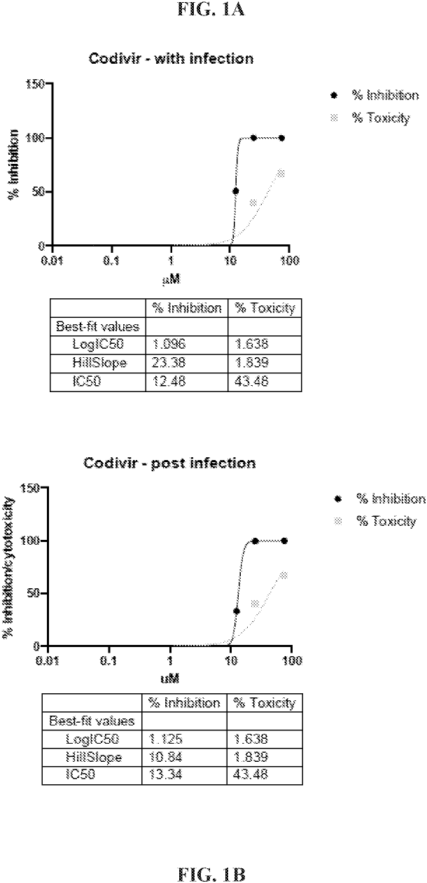

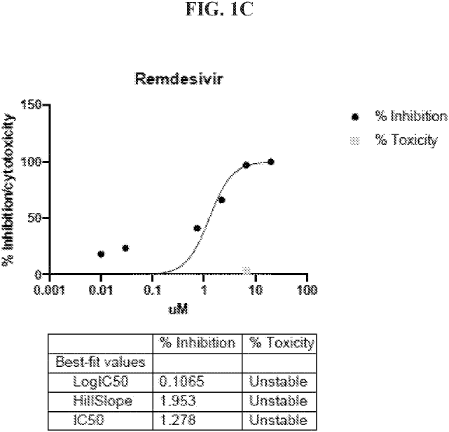

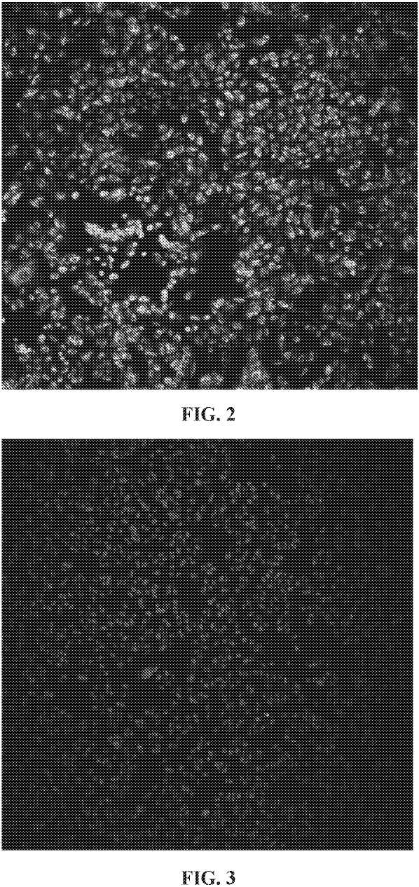

Percentages of infection relative to each concentration, as well as the regression curves for each compound are shown below and in FIGS. 1A-1C.

TABLE 4

EC50, TC50, EC90, TC90, EC99.9, TC99.9 and SI50 (=TC50/EC50) and SI90 (TC90-/EC90) values for each tested compound

| Test article | EC50 (µM) | TC50 (µM) | SI50 | EC90 (µM) | TC90 (µM) | SI90 | EC99.9 (µM) | TC99.9 (µM) |
|---|---|---|---|---|---|---|---|---|
| Codivir - with infection | 12.48 | 43.48 | 3.48 | 13.7 | 143.6 | 10.5 | 16.8 | 185.9 |
| Codivir - post-infection | 13.34 | 43.48 | 3.26 | 16.6 | 143.6 | 8.6 | 25.2 | 185.9 |
| Remdesivir -with infection | 1.28 | ND | >20 µM | 3.9 | ND | >20 µM | 4.4 | ND |

ND: Not determined, due to the inability to extrapolate a curve from the input values.

### Percentages of Infection - IF Assay

Table 5 shows the percentage of Vero cells infected by SARS-CoV2 after incubation with Codivir or Remdesivir (assay control) for 24 h. Eight dilutions were tested as indicated in the table. Three technical replicates were performed for Codivir. Untreated infected and untreated uninfected controls were included.

TABLE 5

Percentage of infection at 24 h

| | | | | | | | | | | | | | | |
|---|---|---|---|---|---|---|---|---|---|---|---|---|---|---|
| (Codivir) uM | (Remd) uM | Codivir - with infection | | | Codivir - post infection | | | Remdeslvir (Remd) | | 0.05% | 0.15% | 0.05% | 0.15% | |
| 75.00 | 20 | 0.06 | 0.08 | 0.03 | 0.02 | 0.09 | 0.12 | 0.09 | 0.04 | 32.29 | 24.35 | 0.04 | 0.06 | |
| 25.00 | 6.67 | 0.06 | 0.04 | 0.02 | 0.18 | 0.09 | 0.09 | 0.59 | 0.43 | 32.12 | 13.93 | 0.09 | 0.04 | Acetic |
| 12.50 | 2.22 | 15.48 | 42.39 | 11.31 | 19.33 | 14.03 | 21.35 | 10.20 | | 12.46 | 55.02 | 0.05 | 0.06 | Acid |
| 4.17 | 0.74 | 48.50 | 24.18 | 63.10 | 34.74 | 44.17 | 8.19 | 8.03 | 9.60 | 4.78 | 43.10 | 0.03 | 0.02 | |
| 1.39 | 0.25 | 61.97 | 16.02 | 7.63 | 59.36 | 35.22 | 20.18 | 41.80 | 23.97 | 4.71 | 13.61 | 0.06 | 0.02 | |
| 0.46 | 0.08 | 27.57 | 33.55 | 29.29 | 22.78 | 30.31 | 36.88 | 4.56 | 27.03 | 13.38 | 21.32 | 0.07 | 0.06 | Suppl. |
| 0.15 | 0.03 | 35.49 | 14.67 | 55.01 | 27.61 | 6.09 | 56.73 | 45.54 | 11.44 | 6.10 | | 0.04 | 0.06 | Media |
| 0.05 | 0.01 | 39.94 | 20.05 | 54.76 | 25.60 | 30.96 | 26.08 | 13.90 | 10.58 | 24.64 | 20.98 | | 0.18 | |

### Percentages of Cytotoxicity

Tables 6 and 7 show the absorbance values (6) and the percentage of cytotoxicity (7) of Vero cells incubated with Codivir or Remdesivir (assay control) for 24 h. For both compound, eight dilutions were tested. Three technical replicates were performed for Codivir. Untreated infected and untreated uninfected controls were included.

TABLE 6

Absorbance values - cytotoxicity at 24 h Codivir Remdesivir

| | | | | | | | | | | | | | | |
|---|---|---|---|---|---|---|---|---|---|---|---|---|---|---|
| uM | uM | Codivir | | | Codivir | | | Remdesivir | | 0.05% | 0.15% | 0.05% | 0.15% | |
| 75.00 | 20 | 0.0407 | 0.0403 | 0.0389 | 0.0414 | 0.0421 | 0.0403 | 0.1234 | 0.1247 | 0.1277 | 0.1126 | 0.1265 | 0.1104 | Acetic Acid |
| 25.00 | 6.67 | 0.0691 | 0.075 | 0.0733 | 0.0763 | 0.0762 | 0.0822 | 0.1198 | 0.1127 | 0.1217 | 0.1269 | 0.1176 | 0.1242 | |
| 12.50 | 2.22 | 0.1196 | 0.1315 | 0.1325 | 0.1324 | 0.1314 | 0.1395 | 0.1599 | 0.1541 | 0.1596 | 0.1578 | 0.1599 | 0.1222 | |
| 4.17 | 0.74 | 0.1306 | 0.1426 | 0.1477 | 0.1432 | 0.1433 | 0.1556 | 0.1606 | 0.1568 | 0.1564 | 0.1529 | 0.1498 | 0.134 | |
| 1.39 | 0.25 | 0.1261 | 0.1377 | 0.139 | 0.1432 | 0.1436 | 0.1453 | 0.1554 | 0.1531 | 0.1394 | 0.1406 | 0.1351 | 0.1152 | Suppl. Media |
| 0.46 | 0.08 | 0.1454 | 0.1447 | 0.1452 | 0.1433 | 0.1468 | 0.1493 | 0.1483 | 0.1462 | 0.1302 | 0.1299 | 0.1225 | 0.1097 | |
| 0.15 | 0.03 | 0.1361 | 0.143 | 0.1487 | 0.1479 | 0.1455 | 0.1446 | 0.147 | 0.1423 | 0.1317 | 0.1324 | 0.12 | 0.1041 | |
| 0.05 | 0.01 | 0.1161 | 0.14563 | 0.1448 | 0.1447 | 0.13.23 | 0.1351 | 0.1378 | 0.1381 | 0.1199 | 0.1126 | 0.1079 | 0.0886 | |

TABLE 7

Percentages of cytotoxicity at 24 h Codivir Remdesivir

| | | | | | | | | | | | | | | |
|---|---|---|---|---|---|---|---|---|---|---|---|---|---|---|
| uM | uM | Codivir | | | Codivir | | | Remdesivir | | 0.05% | 0.15% | 0.05% | 0.15% | |
| 75.00 | 20 | 66.433 | 66.76 | 67.91 | 65.85 | 65.27 | 66.76 | -1.78 | -2.861 | -5.33 | 7.12 | -4.34 | 8.94 | Acetic Acid |
| 25.00 | 6.67 | 43.00 | 38.14 | 39.54 | 37.07 | 37.15 | 32.20 | 1.19 | 7.04 | -0.38 | -4.67 | 3.00 | -2.44 | |
| 12.50 | 2.22 | 1.35 | -8.46 | -9.29 | -9.21 | -8.38 | -15.06 | -31.89 | -27.11 | -31.64 | -30.16 | -31.89 | -0.79 | |
| 4.17 | 0.74 | -7.72 | -17.62 | -21.83 | -18.12 | -18.20 | -28.34 | -32.47 | -19.33 | -29.00 | -26.12 | -23.56 | -10.53 | |
| 1.39 | 0.25 | -4.01 | -13.58 | -14.65 | -18.12 | -18.45 | -19.85 | -28.18 | -26.28 | -14.98 | -15.97 | -11.43 | 4.98 | |
| 0.46 | 0.08 | -19.93 | -19.35 | -19.76 | -18.20 | -21.08 | -23.15 | -22.32 | -20.59 | -7.39 | -7.15 | -1.04 | 9.52 | Suppl. media |
| 0.15 | 0.03 | -12.26 | -17.95 | -22.65 | -21.99 | -20.01 | -19.27 | -21.25 | -17.37 | -8.63 | -9.21 | 1.02 | 14.14 | |
| 0.05 | 0.01 | 4.24 | -20.09 | -19.43 | -19.35 | -9.12 | -11.43 | -13.66 | -13.91 | 1.10 | 7.12 | 11.00 | 26.92 | |

### Percentages of Inhibition and Cytotoxicity: Regression Curves

The graphs in FIGS. 1A and 1B display the percentage of inhibition of SARS-CoV2 infection (black line) at different peptide concentrations. FIG. 1C is a positive control using Remdesivir. Sample values in each plate were normalized to the plate internal controls, where 100% inhibition is derived from the average of the negative control (untreated uninfected) and 0% inhibition is derived from the average of the positive control (untreated infected). The x axes show compound dilutions (uM). The curves represent the best fit of the logarithm of compound dilution vs. the normalized percentage of inhibition (variable slope). Cytotoxicity (toxicity) is displayed in light gray, with values normalized to the plate internal control (untreated cells, 100% viability).

### Conclusion

Under the conditions tested, Codivir displayed antiviral activity against SARS-CoV2 at 24 h post infection, with EC50 values of 12.48 µM-13.34 µM (SI50 of 3.48-3.26) and EC90 values of 13.7 µM-16.6 µM (SI90 10.5-8.6). 90% inhibition of infectivity was reached at concentrations of peptide only marginally higher that the EC50 value; however, at these same concentrations, minimal cytotoxicity was observed, as reflected by a higher selectivity index value.

### Example 3: In Vitro Efficacy of INS Peptide Against Influenza A

This study was performed to determine the cytotoxicity and antiviral activity of the synthetic INS peptide, Codivir, against Influenza A virus (A / WSN /33), when administered at the same time as viral infection, or 2 h after infection.

### Materials and Methods

To determine the antiviral activity of Codivir, MDCK-II cells were either infected with Influenza A virus (IAV) for 2 h, and then treated with a three-fold serial dilution of peptide ranging from 75 µM to 0.05 µM, or treated with the same concentrations of peptide immediately before infection. Antiviral activity was determined at 24 h using an immunofluorescence-based assay. Cytotoxicity was determined using an MTT assay on uninfected cells treated with the same concentrations of peptide. Niclosamide, a broad-spectrum antiviral in vitro, was included as an assay control.

TABLE 8

Test article and stock preparation

| Compound ID | Molecular Weight | Weighted Mass | Diluent | Stock Concentration | Volume of diluent (R1) |
|---|---|---|---|---|---|
| Codivir | 1976.36 | 3 mg | 20% Acetic Acid | 5 mM | 300 |

Stored before reconstitution: -20° C.

Stored after reconstitution: -20° C.

### Controls

Uninfected untreated cells (with 0%, 0.05%, or 0.15% Acetic Acid)
Infected untreated cells (with 0%, 0.05%, or 0.15% Acetic Acid)
Positive control drug: Niclosamide (Sigma, N3510-50G, Lot BCBG9789V)

### Test System

Cells: MDCK-II, p8, (VRS stock from ATCC)
Virus: Influenza A virus (A / WSN /33) (VRS stock p3)
Readout:
   1) High content immunofluorescence
   2) Cell viability (cytotoxicity assays)
Standard Operating Procedures were employed for cell passaging, cell counting, infectivity and cytotoxicity assays; immunofluorescence staining; and the MTT assay.

### Key Reagents

Complete media: DMEM medium (Gibco, 61965059) supplemented with 10% FBS (Gibco 10500064) and 1X p/s (Gibco 15070063)
Supplemented media: DMEM medium (Gibco, 61965059) supplemented with 0.3% BSA (Gibco 15260037), 0.1% FBS (Gibco 10500064), 10 mM Hepes (Gibco, 15630080) and 1X p/s (Gibco 15070063)
Diluent: Acetic Acid (Analar Normapur, VWR 20104.334, Lot 13E030508); 20%:
2m1 Ac. Acid in 8m1 water
Niclosamide (Sigma, N3510-50G, Lot BCBG9789V)

### Experimental Procedure

The antiviral activity of 8 dilutions of Codivir against IAV was explored following two modes of administration: at the same time as infection with virus (with infection), and 2 h after infection with virus (post-infection). Virus and peptide remained in contact with the cells for the entire duration of the experiment (24 h). The cytotoxicity of the same range of concentrations of Codivir was determined by MTT assay.

### Cell Plating

Cells were detached and counted and cell count was recorded in a Cell Count Logbook. Cells were seeded in complete media at 9,000 cells/1001/well in two plates: one for the cytotoxicity assay and one for the infectivity assay. After seeding, the plates were incubated at room temperature (RT) for 5 minutes for even distribution, and then at 37° C., 5% CO, until the following day.

### Infection

The virus stock was diluted 10-fold to bring the concentration to 1×10⁶ TCID50 /ml. 2.8 µl of diluted virus were transferred into 4,397 \.2 µl of supplemented media (Multiplicity of Infection (MOI) 0.002). Media was removed from the cells and 50 µl of virus added in columns 1-10. 50 µl of supplemented media without virus were added in columns 11-12 (uninfected control).

### Peptide Preparation

Two peptide stocks were prepared:
1) 150 µM stock
2) 25 µM stock

The initial 5 mg/ ml (— 5 mM, 20% Acetic Acid) Codivir stock was prepared as presented in Table 8. 300µl of water were added to bring the stock to 2.5 mM, 10% Acetic Acid. 78µl of 2.5 mM peptide were added to 1.222µl of supplemented media to reach the 150 µM concentration. 13µl of 2.5 mM peptide were added to 1.287µl of supplemented media to reach the 25 µM concentration.

In order to preserve the same final concentration of Acetic Acid across the serial dilution (and no lower than 0.05%), three assay media were prepared:
1) supplemented media containing no Acetic Acid (“supplemented media”)
2) supplemented media containing the same concentration of Acetic Acid as the top dilutions of the 25 µM concentration (0.1%; “supplemented media + diluent 0.1%” → 12 ml + 12µl 100% Acetic Acid)
3) supplemented media containing the same concentration of Acetic Acid as the top dilution of the 150 µM concentration (0.3%; “supplemented media + diluent 0.3%” → 12 ml + 36µl 100% Acetic Acid)
   120µl of “supplemented media + diluent 0.1%” were added in each row of column 1-6 of a round-bottom 96 well plate, except rows A, B, C.
In row B, 120µl of “supplemented media” (no diluent) were added.

180µl of peptide at 150 µM were added to wells 1 to 6 of row A, and diluted 3-fold by transferring 6041 into row B.

180µl of peptide at 25 uM were added to wells 1 to 6 in row C, and serially diluted down (3-fold) by transferring 6041 to the next rows.

120µl of “Supplemented media + diluent 0.1%” were added into columns 9 and 11, rows A to D.

120µl of “supplemented media + diluent 0.3%” were added into columns 10 and 12, rows A to D.

120µl of “supplemented media” were added into columns 9 to 12, rows E to H, to control for the effect of Acetic Acid on cells and virus.

1.3µl of a 10 mM stock or Niclosamide in DMSO were added to 698.741 of supplemented media (20 µM).

180µl of 20 µM Niclosamide were added to row A of columns 7 and 8

120µl of supplemented media were added in the remaining rows.

60µl of 20 µM Niclosamide were moved down in a 3-fold dilution series.

### Peptide Treatment

Either immediately after infection or 2 h after infection, 50µl of treatment media from the dilution plate were transferred to the cells in each plate (infectivity and cytotoxicity). Niclosamide was added immediately after infection. 50µl of media without diluent were added to the cytotoxicity plate instead of virus. Both plates were incubated at 37° C., 5% CO, for 24 h.

### Fixation and Development

After 24 h, one plate was washed with PBS, fixed for 30 minutes with 4% formaldehyde, washed again with PBS, and stored in PBS at 4° C. until staining. The cytotoxicity plate was treated with MTT to determine cell viability.

### Infectivity Readout

Cells were immunostained. Briefly, any residual formaldehyde was quenched with 50 mM ammonium chloride, after which cells were permeabilized (0.1% Triton X100) and stained with an antibody recognizing IAV nucleoprotein (GeneTex GTX14213). The primary antibody was detected with an Alexa-488 conjugate secondary antibody (Life Technologies, A11001), and nuclei were stained with Hoechst. Images were acquired on an Opera Phenix high content confocal microscope (Perkin Elmer) using a 10X objective, and percentage infection calculated using Columbus software (infected cells/total cells x 100).

### Cytotoxicity Readout

Cytotoxicity was detected by MTT assay using standard protocols. Briefly, the MTT reagent (Sigma, M5655) was added to the cells for 2 h at 37° C., 5% CO, after which the media was removed and the precipitate solubilized with a mixture of 1:1 Isopropanol:DMSO for 20 minutes. The supernatant was transferred to a clean plate and signal read at 570 nm.

### Determination of EC50 Concentration — IF Assay

Normalized percentages of inhibition were calculated using the following formula:

$$\begin{matrix}N o r m a l i z e d \% i n h i b i t i o n = 100 \times \\ ( 1 - \frac{\% I n f e c t i o n S a m p l e \text{-} \% I n f e c t i o n U n i n f e c t e d C o n t r o l )}{\% I n f e c t i o n I n f e c t e d C o n t r o l \text{-} \% I n f e c t i o n U n i n f e c t e d C o n t r o l}\end{matrix}$$

EC50 values were extrapolated from the curves representing the best fit (non-linear regression analysis, variable slope) of the logarithm of compound concentration vs. the normalized percentages of inhibition, using GraphPad Prism (version 9).

### Determination of TC50 Concentration

Percentages of cytotoxicity were calculated using the following formula:

$$\% c y t o t o x i c i t y = 100 - ( 100 \text{x} \frac{A b s o r b a n c e S a m p l e )}{A b s o r b a n c e U n t r e a t e d C o n t r o l}$$

TC50 values were extrapolated from the curves representing the best fit (non-linear regression analysis, variable slope) of the logarithm of compound concentration vs. the normalized percentages of cytotoxicity, using GraphPad Prism (version 9).

### Summary of Results

Using an immunofluorescence-based assay at 24 h after infection, Codivir showed antiviral activity against IAV with an EC50 of 9.28 µM when administered at the same time as infection and 29.55 µM when administered post infection. The selectivity index was 5.31 and 1.73, respectively. The corresponding EC90 values were 79.28 µM and 71.62 µM, respectively. No significant cytotoxicity or antiviral activity was observed in the presence of vehicle (acetic acid) only.

Table 9 displays the EC50, EC90, TC50, TC90 and Selectivity Index (SI) 50 and SI90 for Codivir and the Niclosamide control. Inhibition of IAV infection was observed in cells treated with Codivir, with EC50 of 9.281 uM (peptide administered at the same time as the virus) and 29.55 µM (peptide administered post infection). EC90 for the same experimental conditions was 79.29 µM and 71.62 µM, respectively. Some cytotoxicity was observed at the highest concentrations tested, with a TC50 value around 50 µM, and a TC90 value around 100 µM. The SI50 was 5.31 when the peptide was administered at the same time as the infection, and 1.73 when it was administered after infection. The respective SI90 values are 1.27 and 1.35.

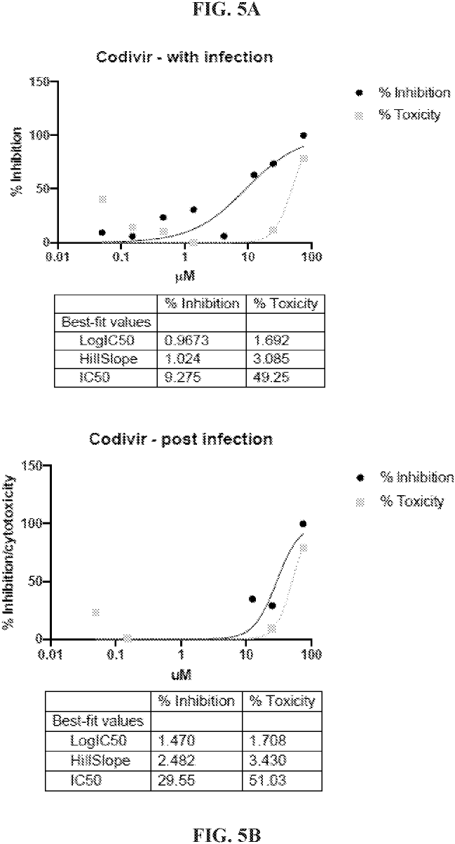

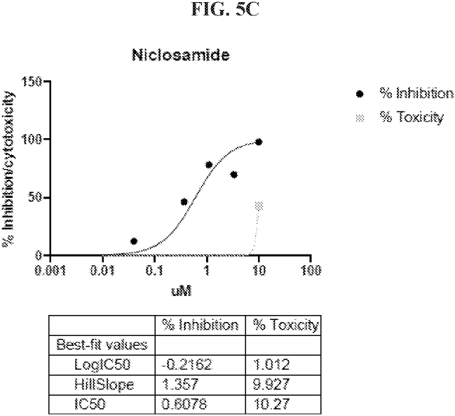

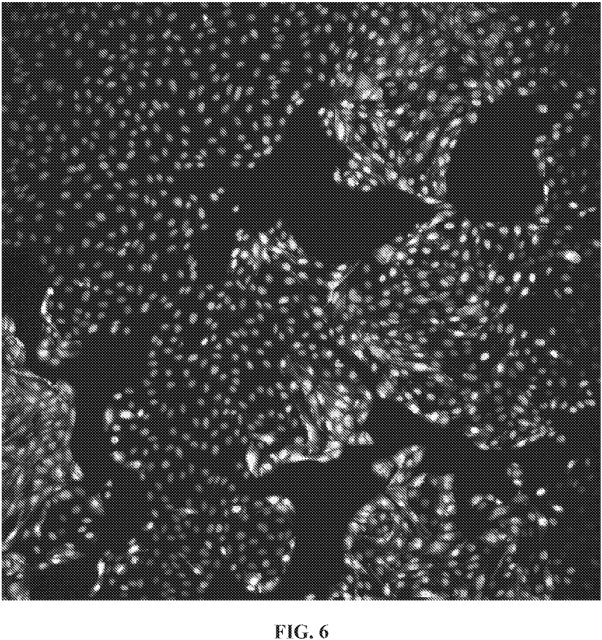

Percentages of infection relative to each concentration, as well as the regression curves for each compound are shown in FIGS. 5A-5C.

TABLE 9

EC50, TC50, EC90, TC90, and 8150 (=TC50/EC50) and 8190 (=TC90 /EC90) values for each tested compound

| Test article | EC50 (RA) | TC50 (riM) | SI50 | EC90 (RA) | TC90 (riM) | SI90 |
|---|---|---|---|---|---|---|
| Codivir — with infection | 9.28 | 49.25 | 5.31 | 79.28 | 100.39 | 1.27 |
| Codivir — post-infection | 29.55 | 51.03 | 1.73 | 71.62 | 96.83 | 1.35 |
| Niclosamide — with infection | 0.61 | 10.27 | 16.84 | 3.06 | 12.81 | 4.19 |

### Percentages of Infection - IF Assay

Table 10 shows the percentage of MDCK-II cells infected by IAV after incubation with Codivir or Niclosamide (assay control) for 24 h. Eight dilutions were tested as indicated in the table. Three technical replicates were performed for Codivir. Untreated infected and untreated uninfected controls were included.

TABLE 10

Percentages of infection at 24 h

| (Codivir) µM | (Niclos) µM | Codivir - with infection | | | Codivir - post infection | | | Niclosamide (Niclos) | | 0.05% | 0.15% | 0.05% | 0.15% | |
|---|---|---|---|---|---|---|---|---|---|---|---|---|---|---|
| 75.00 | 10.00 | 0.03 | 0.03 | 0.08 | 0.05 | 0.05 | 0.12 | 0.20 | 0.21 | 12.54 | 24.00 | 0.01 | 0.01 | Acetic Acid |
| 25.00 | 3.33 | 2.31 | 10.96 | 1.92 | 14.76 | 11.56 | 14.42 | 2.33 | 3.18 | 23.19 | 23.52 | 0.06 | 0.01 | |
| 12.50 | 1.11 | 10.43 | 7.98 | 2.85 | 11.34 | 17.82 | 8.29 | 1.84 | 2.12 | 22.59 | 9.01 | 0.02 | 0.01 | |
| 4.17 | 0.37 | 24.64 | 20.19 | 9.19 | 40.88 | 25.09 | 23.94 | 4.87 | 15.48 | 3.41 | 35.19 | 0.00 | 0.02 | Suppl. Media |
| 1.39 | 0.12 | 9.65 | 12.20 | 17.92 | 35.96 | 12.95 | 14.27 | 8.15 | 11.55 | 6.65 | 9.91 | 0.00 | 0.01 | |
| 0.46 | 0.04 | 13.38 | 19.61 | 11.00 | 12.86 | 9.06 | 43.96 | 5.89 | 10.02 | 15.03 | 6.01 | 0.01 | 0.02 | |
| 0.15 | 0.01 | 27.75 | 11.53 | 14.86 | 24.95 | 30.48 | 32.08 | 11.78 | 11.17 | 8.04 | 6.65 | 0.02 | 0.00 | |
| 0.05 | 0.00 | 16.01 | 24.62 | 11.44 | 36.39 | 29.03 | 22.41 | 13.13 | 11.26 | | 11.11 | 0.01 | 0.00 | |

### Percentages of Cytotoxicity

Tables 11 and 12 show the absorbance values (11) and the percentage of cytotoxicity (12) of MDCK-II cells incubated with Codivir or Niclosamide (assay control) for 24h. For both compound, eight dilutions were tested. Three technical replicates were performed for Codivir. Untreated infected and untreated uninfected controls were included.

TABLE 11

Absorbance values - cytotoxicity at 24 h Codivir Niclosamide

| | | | | | | | | | | | | | | |
|---|---|---|---|---|---|---|---|---|---|---|---|---|---|---|
| µM | µM | Codivir | | | Codivir | | | Niclosamide | | 0.05% | 0.15% | 0.05% | 0.15% | |
| 75.00 | 10 | 0.0422 | 0.0436 | 0.0425 | 0.0421 | 0.0421 | 0.0419 | 0.107 | 0.1166 | 0.1014 | 0.0979 | 0.1013 | 0.1003 | Acetic Acid |
| 25.00 | 3.33 | 0.1794 | 0.1713 | 0.1708 | 0.1681 | 0.1845 | 0.1841 | 0.2005 | 0.1965 | 0.2206 | 0.1796 | 0.1876 | 0.1522 | |
| 12.50 | 1.11 | 0.183 | 0.2158 | 0.2057 | 0.2165 | 0.2045 | 0.2113 | 0.227 | 0.2465 | 0.2136 | 0.1862 | 0.2125 | 0.1673 | |
| 4.17 | 0.37 | 0.2071 | 0.1982 | 0.2203 | 0.2153 | 0.2027 | 0.2054 | 0.2429 | 0.2319 | 0.1965 | 0.1583 | 0.1861 | 0.1485 | |
| 1.39 | 0.12 | 0.1789 | 0.1944 | 0.2184 | 0.2175 | 0.2126 | 0.2174 | 0.213 | 0.2407 | 0.2326 | 0.2237 | 0.2221 | 0.2037 | Suppl. Media |
| 0.46 | 0.04 | 0.1522 | 0.1679 | 0.2104 | 0.2062 | 0.1922 | 0.1978 | 0.2247 | 0.2292 | 0.2048 | 0.2282 | 0.2121 | 0.192 | |
| 0.15 | 0.01 | 0.1552 | 0.1707 | 0.1789 | 0.1965 | 0.1896 | 0.2019 | 0.2048 | 0.2048 | 0.1745 | 0.1926 | 0.1917 | 0.162 | |
| 0.05 | 0 | 0.0985 | 0.1183 | 0.1389 | 0.1493 | 0.1468 | 0.1612 | 0.1646 | 0.1811 | 0.2153 | 0.1981 | 0.1581 | 0.1457 | |

TABLE 12

Percentages of cytotoxicity at 24 h

| | | | | | | | | | | | | | | |
|---|---|---|---|---|---|---|---|---|---|---|---|---|---|---|
| Codivir µM | Niclos µM | Codivir - with infection | | | Codivir - post infection | | | Niclosamide (Niclos) | | 0.05% | 0.15% | 0.05% | 0.15% | |
| 75.00 | 10 | 78.61 | 77.9 | 78.46 | 78.66 | 78.66 | 78.77 | 45.77 | 40.91 | 48.61 | 50.39 | 48.66 | 49.17 | Acetic Acid |
| 25.00 | 3.33 | 9.08 | 13.19 | 13.44 | 14.81 | 6.5 | 6.7 | -1.61 | 0.42 | -11.8 | 8.98 | 4.93 | 22.87 | |
| 12.50 | 1.11 | 7.26 | -9.36 | -4.24 | -9.72 | -3.64 | -7.08 | -15.04 | -24.92 | -8.25 | 5.64 | -7.69 | 15.22 | |
| 4.17 | 0.37 | -4.95 | -0.44 | -11.64 | -9.11 | -2.72 | -4.09 | -23.1 | -17.52 | 0.42 | 19.78 | 5.69 | 24.74 | |
| 1.39 | 0.12 | 9.34 | 1.48 | -10.68 | -10.22 | -7.74 | -10.17 | -7.94 | -21.98 | -17.88 | -13.37 | -12.56 | -3.23 | Suppl. media |
| 0.46 | 0.04 | 22.87 | 14.91 | -6.63 | -4.5 | 2.6 | -0.24 | -13.87 | -16.15 | -3.79 | -15.65 | -7.49 | 2.7 | |
| 0.15 | 0.01 | 21.35 | 13.49 | 9.34 | 0.42 | 3.91 | -2.32 | -3.79 | -3.79 | 11.57 | 2.39 | 2.85 | 17.9 | |
| 0.05 | 0 | 50.08 | 40.05 | 29.61 | 24.34 | 25.6 | 18.31 | 16.58 | 8.22 | -9.11 | -0.39 | 19.88 | 26.16 | |

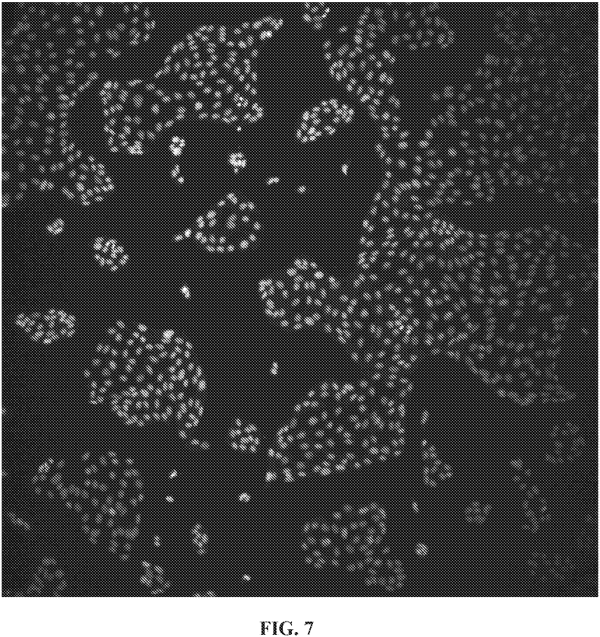

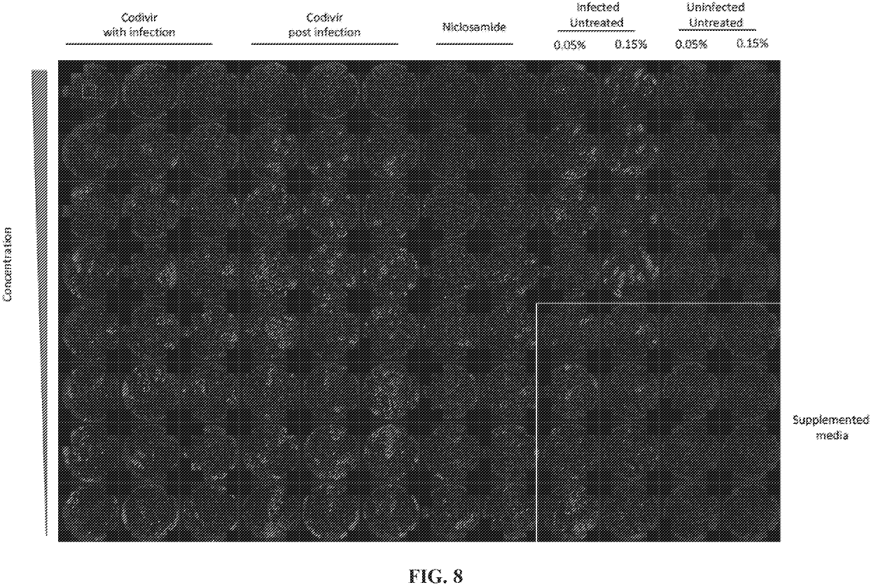

The graphs in FIGS. 5A and 5B present the percentage of inhibition of IAV infection (black line) at different peptide (Codivir) concentrations. Sample values in each plate were normalized to the plate internal controls, where 100% inhibition is derived from the average of the negative control (untreated uninfected) and 0% inhibition is derived from the average of the positive control (untreated infected). FIG. 5C shows a positive control using Niclosamide (Niclos). The x axes show compound dilutions (µM). The curves represent the best fit of the logarithm of compound (Codivir or positive control, Niclosamide) dilution vs. the normalized percentage of inhibition (variable slope). Cytotoxicity is displayed in gray, with values normalized to the plate internal control (untreated cells, 100% viability). FIG. 6 is a representative image of untreated infected cells (0.05% Acetic Acid). Light gray: infected cells, stained for the virus nucleoprotein; dark gray: total cells, nuclei staining. FIG. 7 is a representative image of untreated uninfected cells (0.05% Acetic Acid). Dark gray: total cells, nuclei staining. FIG. 8 is an overview of the assay plate. Light gray: infected cells, stained for the virus nucleoprotein; dark gray: total cells, nuclei staining.

### Conclusions

Under the conditions tested, Codivir displayed antiviral activity against IAV at 24 h post infection, with EC50 values of 9.28 µM when administered at the same time as the infection, and 29.551 M when administered 2 h after infection, with higher potency in the first mode of administration (at the same time as infection) for this virus. EC90 values were closer between the two modes of administration, 79.28 µM and 71.62 µM respectively.

### Example 4 -- Study of Codivir in Outpatients With COVID-19

Described in this example is an open label study to evaluate the safety and preliminary efficacy of Codivir in 12 mild or moderate COVID-19 patients and the onset of symptoms within 96 hours (96 h) prior to their inclusion in the study. Treatment commences in the hospital, and the study participants are discharged at Day 4 and continue the treatment up to Day 10 at home. The study compound Codivir (20 mg) is administered by subcutaneous (SC) injection twice a day for 10 days. The study compound, Codivir peptide (Zion Medical, Israel), is a chemically synthesized 16-amino acid peptide formulated as a sterile and non-pyrogenic aqueous solution in a single-use vial containing 10 mg/ml of the product for Sub-Cutaneous (SC) administration. The peptide is produced by Polypeptide USA. The Final Drug Product was produced and released by Zion Medical under sterile laminar hood under Good Manufacturing Practice (GMP) conditions.

Preliminary safety results in pilot studies using human subjects have demonstrated that Codivir is tolerable, exhibits no reported side effects and no harm to immune system, with no adverse events, meeting the primary endpoint of the studies. No adverse events or severe adverse events were detected. Vital signs, physical examinations exhibited no significant differences in respect for the new treatment. Also, blood tests showed no significant changes relative to the baseline.

### Study Procedure

This open study is designed to evaluate the safety and collect preliminary efficacy data for Codivir administered to 12 adults with COVID-19. Flu symptoms (fever, cough, myalgia and changes in smell or taste) onset are exhibited in the subjects within 96 hours prior to inclusion in the study. Eligible participants who agree to participate and provide their consent are submitted to safety assessments, an RT-PCR and a quick test for COVID-19. The result of the rapid quick test allows for inclusion in the study.

All participants are hospitalized and receive the treatments indicated for their medical condition, except for other investigational medications. Codivir is administered in addition to the treatment at a dose of 20 mg / SC twice a day. The second day is expected to confirm the diagnosis of COVID-19 by RT-PCR. If the result is negative, the participant is discontinued and considered an inclusion failure. If positive, he/she remains hospitalized for 3 days. At the fourth day, in case of improvement, participant is discharged from the hospital and continues the treatment up to Day 10 at home. A nurse visits the participant twice a day to administer Codivir and collect vital signs. Participants are followed up to the 28th day by telemedicine. A doctor calls periodically to monitor the clinical evolution, collect adverse events, concomitant medication and instruct the participant. In case of unfavorable evolution, the participants remain hospitalized and receive the appropriate care. The investigator determines whether or not the investigational medication continues, considering the participant’s health and well-being.

### Study Population

The study population includes patients (adults aged ≥18 years) with mild or moderate COVID-19 and flu-symptoms onset within 96 hours prior to inclusion. Treatment begins at the hospital; on the 4th day, participants who are well are discharged and continue treatment up to Day 10 at home. All participants receive Codivir (20 mg), SC, 2 times daily.

The inclusion criteria for enrollment in this particular study include, without intending to be limiting: age between 18 and 60 years; male or female; SARS-CoV-2 infection indicated by rapid test and confirmed by RT-PCR; mild or moderate COVID-19: oxygen saturation in ambient air >93%; -<30 breaths per minute; no signs of hemodynamic decompensation; absence of pregnancy in women of childbearing age; ability to understand and comply with the requirements of the protocol; and consent to participate. The exclusion criteria for enrollment in this particular study include, without intending to be limiting: participants in need of O₂ supplementation by catheter or mask, invasive mechanical ventilation, or vasopressors; onset of symptoms or rapid test or positive RT-PCR for more than 72 hours of inclusion; participants in use or expected to use within 24 hours prior to the inclusion of drugs that are under clinical investigation as a therapeutic option for the treatment of COVID-19 (e.g., hydroxychloroquine, chloroquine, ivermectin, nitazoxanide, among others) during the period study duration; body mass index less than 19.9 or greater than 35; comorbidities such as, e.g., other serious infections, active malignancies, autoimmune diseases, liver, kidney or heart failure; another systemic disease and / or laboratory abnormality, which, in the investigator’s opinion, prevent the patient from participating in the study; concomitant HIV, HBV or HCV infection; pregnancy or lactation; participation in another clinical trial in the 12 months preceding inclusion; anti-COVID-19 vaccination at any time; vaccination for any other infection in the 4 weeks prior to inclusion; any condition that increases the risk of participating in the study, in the opinion of the investigator. It will be appreciated by the skilled practitioner that such inclusion and exclusion criteria are particular for the study described herein, and are not intended to limit in any way the administration of the compound to a diversity of participants in other studies, regardless of the criteria selected in this for inclusion or exclusion in this particular study.

### Study Objectives

The primary objective of the study described in this example is to evaluate the safety of Codivir peptide administration in mild or moderate COVID-19 patients The secondary objective of the study is to evaluate preliminary efficacy data of the Codivir peptide in participants with COVID-19. The primary endpoint of the study are incidence and severity of adverse events related to the investigational product. The secondary endpoints of the study are clinical evolution according to the scores of the World Health Organization and NEWS2 between admission and days 10 and 28; a negative RT-PCR assessment; and evolution of IgM and IgG anti-SARS-CoV-2 antibodies.

### Screening of Study Participants

Participants with flu symptoms onset within 96 hours prior to the study visit are evaluated according to eligibility and invited to participate in the study. Following informed consent, study participants undergo a complete medical evaluation, including anamnesis, personal history and physical examination, collection of anthropometric data (weight and height) and vital signs (CF, BP, RF, T) and O2 saturation. A quick test for COVID-19 (One Step Test Wondfo), an electrocardiogram and screening tests are performed, including serology and pregnancy test for non-sterile women.

### Screening Visit (V-1)

At the screening visit, the following procedures or activities occur: evaluation of the inclusion and exclusion and discontinuation criteria; application, clarification and signing of the Informed Consent Form; conducting the rapid test for the diagnosis of COVID-19 based on the detection of anti-SARS-CoV-2 antibodies in the blood; collection of RT-PCR SARS-CoV-2 for detection of viral RNA in nasopharyngeal and oropharyngeal samples using known Real Time Protein Chain Reaction (RT-PCR) techniques; collection of screening tests complete blood count, urea, creatinine, uric acid, sodium, potassium, chloride, calcium, glucose, glycated hemoglobin, alkaline phosphatase (FA), alanine aminotransferase (ALT), aspartate aminotransferase (AST), total bilirubins and fractions (BTF), total proteins and fractions (PTF), lipid profile (total cholesterol, fractions and triglycerides); coagulogram (TP, aPTT); D-dimer; C-reactive protein; HIV, HBV, HCV serology; Ferritin; pregnancy test for non-sterile women; performing an electrocardiogram in 12 leads with a report; medical evaluation: face-to-face evaluation by the principal investigator or assistant physician with a complete physical examination; evaluation of the NEWS-2 score; evaluation of the World Health Organization score; and collection of data on concomitant medication.

Visit 0 -- First drug administration. Procedures and activities at visit include: medical evaluation; confirmation of eligibility by the inclusion, exclusion and discontinuation criteria; evaluation of the NEWS-2 score; evaluation of the World Health Organization score; collection of data on concomitant medication.; first administration of the investigational product Codivir at a dose of 20 mg twice a day subcutaneously; and collection of information on concomitant medication and adverse events.

Visits 1 and 2 -- Scheduled period of hospitalization and hospital discharge. Procedures and activities at visit include: medical evaluation; confirmation of eligibility by the inclusion, exclusion and discontinuation criteria; collection of periodic tests: complete blood count, urea, creatinine, uric acid, sodium, potassium, chloride, calcium, glucose, alkaline phosphatase (FA), alanine aminotransferase (ALT), aspartate aminotransferase (AST), total bilirubins and fractions (BTF) , total proteins and fractions (PTF), lipid profile (total cholesterol, fractions and triglycerides); coagulogram (TP TTPa); D-dimer; C-reactive protein; Ferritin; fibrinogen; administration of the investigational product CODIVIR at a dose of 20 mg twice a day subcutaneously. The first dose of the day is administered in hospital and the second at home; evaluation of the NEWS-2 score; evaluation of the World Health Organization score; collection of data on concomitant medication and adverse events; and hospital discharge if participants are well (V2).

Visits 3, 4, 5, 6, 7, 8, 9 -- Scheduled follow-up period for home treatment. Procedures and activities include: home visits by a nurse; administration of the investigational product Codivir at a dose of 20 mg twice a day subcutaneously; evaluation of the NEWS-2 score; evaluation of the World Health Organization score; collection of periodic examinations: complete blood count, creatinine; alkaline phosphatase (FA), alanine aminotransferase (ALT), aspartate aminotransferase (AST); Dimer d; C-reactive protein; collection of data on concomitant medication and adverse events; telephone contact by a doctor to monitor the evolution, and guide the participant and collect information on concomitant medication and adverse events (V4 to V9).

Post-Treatment Follow-up -- After the end of the treatment, study participants continue to be monitored as described in the following visits.

Visit 10 -- Return of the participant for on-site medical evaluation, examinations and medical evaluation. Procedures and activities include: evaluation of the NEWS-2 score; evaluation of the World Health Organization score; collection of periodic examinations: complete blood count, creatinine; alkaline phosphatase (FA), alanine aminotransferase (ALT), aspartate aminotransferase (AST); Dimer d; C-reactive protein; performing an electrocardiogram in 12 leads with a report; collection of data on concomitant medication and adverse events; COVID-19 test: SARS-CoV-2 (COVID-19) - RT-PCR; and IgM and IgG anti-SARS-CoV-2 antibody titer analysis.

Visits 11, 12 and 13 -- Home care. Procedures and activities include: medical evaluation by telemedicine; evaluation of the NEWS-2 score; evaluation of the World Health Organization score; collection of data on concomitant medication; and collection of information about adverse events.

Visit 14 -- Termination of participation in the study. Procedures and activities include: medical evaluation; collection of final exams: complete blood count, creatinine, sodium, potassium, chloride, calcium, glucose, alkaline phosphatase (FA), alanine aminotransferase (ALT), aspartate aminotransferase (AST), total bilirubins and fractions (BTF), total proteins and fractions (PTF), lipid profile (total cholesterol, fractions and triglycerides); coagulogram (TP, aPTT); D-dimer; C-reactive protein; conducting an electrocardiogram (ECG); RT-PCR collection for SARS-CoV-2; blood collection to measure IgG and IgM anti SARS CoV-2 immunoglobulin titer; evaluation of the World Health Organization score; collection of data on concomitant medication; and collection of information about adverse events.

Extra Visit -- In case of an extra visit, such a visit is carried out at the participant’s initiative or medical recommendation, including at least the following: medical evaluation; collection of blood for periodic examinations: complete blood count, creatinine; alkaline phosphatase (FA), alanine aminotransferase (ALT), aspartate aminotransferase (AST); Dimer d; C-reactive protein; evaluation of the NEWS-2 score; evaluation of the World Health Organization score; collection of data on concomitant medication; and collection of information about adverse events.

For this study, enrollment failure is defined as a subject (participant) who initiates the study (i.e., Visit 2) but who cannot initiate drug therapy for any reason and is therefore withdrawn from the study. If a subject is withdrawn from the study after initiating therapy (is allowed no further study visits), reasonable efforts are made complete all study procedures. The reason(s) for a subject’s withdrawal from the study are recorded in the source documentation and on the CRF, and the subject is not used in the primary analyses. The Investigator makes reasonable attempts to contact subjects who are lost to follow-up (e.g., a minimum of two documented phone calls and a registered letter with return receipt are considered reasonable).

An adverse event (AE) encountered in this study is any untoward sign (including an abnormal laboratory finding), symptom or disease temporally associated with the use of a medical product whether or not considered as related to the medical product, minor or serious. A new condition or the worsening of a chronic or intermittent pre-existing condition are considered to be a possible AE. Any abnormal laboratory findings or diagnostic procedures (vital signs, physical exam, ECG, etc.) considered to be clinically significant are recorded as possible AEs. Events not meeting the definition of AE include a change in clinical status including laboratory findings or other safety parameters which are associated with the investigated disease and are part of its possible or expected progression, unless more severe than expected as judged by the investigator.

Standard of care (SOC) medications are allowed during the study. For example, Remdesivir and glucocorticosteroids or plasma treatment are allowed during the study. All concomitant medications used are recorded in the subject’s medical file and on the appropriate CRF page.

The clinical study results are utilized to evaluate the safety and the preliminary efficacy of Codivir administration in patients (n=12) who have mild or moderate COVID-19 disease and the onset of symptoms within 96 hours prior to their inclusion. Treatment with Codivir begins in the hospital; thereafter, participants are discharged at Day 4 and continue the treatment up to Day 10 at home. The reduction, diminution, amelioration, abatement, or elimination of Covid-19 virus, disease, and/or symptoms thereof following treatment with Codivir during the study provide endpoints that are associated with beneficial and advantageous therapeutic use of the compound.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment or an aspect herein includes that embodiment or aspect as any single embodiment or in combination with any other embodiments or portions thereof.

All patents and publications mentioned in this specification are herein incorporated by reference to the same extent as if each independent patent and publication was specifically and individually indicated to be incorporated by reference.

Although the present invention has been described herein above by way of preferred embodiments thereof, it can be modified, without departing from the spirit and nature of the subject invention as defined in the appended claims.

1. A method of treating a corona or influenza virus infection or disease in a subject, the method comprising administering to the subject an effective amount of a pharmaceutical composition comprising a peptide comprising the amino acid sequence WTAVQMAVFIHNFKRK (SEQ ID NO: 1), or a fragment, derivative or analog thereof.

2. The method of claim 1, wherein the coronavirus is selected from MERS-CoV, SARS-CoV and SARS-CoV-2.

3. The method of claim 2, wherein the disease is COVID-19.

4. The method of claim 1, wherein the influenza virus is influenza A virus.

5. The method of claim 1, wherein the pharmaceutical composition comprises from about 0.1 to about 30 mg/ml of hydrochloride salt of the peptide (INS-HCl).

6. The method of claim 1, wherein the pharmaceutical composition comprises from about 0.1 wt% to about 15 wt% of a poloxamer having the structure of Formula I, 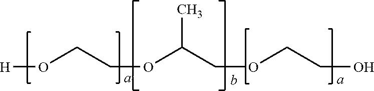
wherein the pH of the composition is between 4 to about 7.5 and wherein α is an integer from 50 to 120, and b is an integer from 15 to 40.

7. The method of claim 6, wherein α is an integer from 60 to 90 and b is an integer from 25 to 35.

8. The method of claim 7, wherein α=80 and b=27 (poloxamer 188).

9. The method of claim 1, wherein the pharmaceutical composition comprises from about 0.1 wt% to about 5 wt% of poloxamer 188.

10. The method of claim 1, wherein the pharmaceutical composition further comprises a saccharide in an amount of about 1 wt% to about 20 wt%.

11-13. (canceled)

14. The method of claim 10, wherein the pharmaceutical composition comprises a saccharide selected from mannitol, lactose, or both mannitol and lactose.

15. The method of claim 14, wherein the pharmaceutical composition comprises D-mannitol.

16. The method of claim 15, wherein the pharmaceutical composition comprises from 5 to 15 mg/ml of INS-HC1, from about 0.1 to about 5 wt% of poloxamer 188, and from about 1 to about 10 wt% of mannitol.

17. The method of claim 16, wherein the pharmaceutical composition comprises from 8 to 12 mg/ml of INS-HCl, from about 0.3 to about 1 wt% of poloxamer 188, and from about 3 to about 6 wt% of mannitol.

18. The method of claim 1, wherein the pharmaceutical composition is administered subcutaneously, intravenously, or intramuscularly.

19. The method of claim 18, wherein the pharmaceutical composition is administered subcutaneously.

20. The method of claim 5, wherein from 5 to 100 mg/day of INS-HCl is administered to the subject.

21. The method of claim 20, wherein from 10 to 60 mg/day of INS-HCl is administered to the subject.

22. The method of claim 1, further comprising administering an additional active agent to the subject.

23. The method of claim 22, wherein the additional active agent is an antiviral active agent.

24-56. (canceled)
